(19) United States
(12) Patent Application Publication
(10) Pub. No.: US 2026/0148807 A1
(43) Pub. Date: May 28, 2026

(54) DETERMINING A CONDITION USING FRAGMENTOMIC ENDPOINT DATA

(71) Applicant: Foundation Medicine, Inc., Cambridge, MA (US)

(72) Inventors: Ethan S. Sokol, Somerville, MA (US); Zoe R. Fleischmann, Jamaica Plain, MA (US); Alexander Fine, Cambridge, MA (US); Brennan Decker, Winchester, MA (US); Kevin Cabrera, Boston, MA (US); Brian Giacopelli, Medford, MA (US); Cai John, Somerville, MA (US); Jie He, Newton, MA (US); Zheng Kuang, Cambridge, MA (US)

(21) Appl. No.: 19/396,917

(22) Filed: Nov. 21, 2025

Related U.S. Application Data
(60) Provisional application No. 63/723,830, filed on Nov. 22, 2024.
(60) Provisional application No. 63/723,846, filed on Nov. 22, 2024.
(60) Provisional application No. 63/868,215, filed on Aug. 21, 2025.

(51) Int. Cl. G16B 30/20; C12Q 1/6869; G16B 40/20; G16H 20/10; G16H 50/30

(52) U.S. Cl. CPC G16B 30/20; C12Q 1/6869; G16B 40/20; G16H 20/10; G16H 50/30

(57) ABSTRACT

Techniques for identifying a condition, such as a tumor classification, of a subject are described. In an example method, sequence read data of a sample obtained from the subject is identified. The sequence read data is indicative of endpoint positions of nucleic acid molecules in the sample. The example method further comprises determining endpoint positions of the nucleic acid molecules, generating input features based on the endpoint positions of the nucleic acid molecules, and classifying, using a classifier, the condition of the subject based on the input features.

### CROSS-REFERENCE TO RELATED APPLICATION

This application claims priority to U.S. Provisional Application No. 63/723,830 filed on Nov. 22, 2024, U.S. Provisional Application No. 63/723,846 filed on Nov. 22, 2024, and U.S. Provisional Application No. 63/868,215 filed on Aug. 21, 2025, which are incorporated by reference herein in their entirety

### BACKGROUND

Many individuals rely on genetic testing to identify whether they have, or are predicted to develop, various health related conditions. Genetic testing can be used to identify sequences that are indicative of a particular genetic disorder or a propensity for disease. In some cases, whole exome sequencing (WES) and whole genome sequencing (WGS) can be used to gain greater context into an individual's health.

Extensive genomic sequencing methodologies, such as those utilizing sequence read data obtained by WGS, can result in a substantial amount of data for analysis. It may be difficult to process this substantial amount of data, directly, to accurately identify whether an individual has a particular condition, such as a type of cancer. For instance, a substantial amount of processing resources may be utilized in order to identify a condition of a subject by analyzing sequences of nucleic acid molecules indicated by the sequence read data. Moreover, some conditions are not apparent when evaluating sequence read data directly.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates an example environment for predicting a condition of a subject based on fragmentomic features of the subject.

FIG. 2 illustrates example preprocessing of fragmentomic data for use in health-related condition classification.

FIG. 3 illustrates an example environment for training and utilizing a predictive model to identify a condition of a subject.

FIG. 4 illustrates an example of training data utilized to train one or more machine learning (ML) models.

FIG. 5 illustrates an example report summarizing predicted conditions of a subject.

FIG. 6 illustrates an example environment for sequencing various nucleic acid molecules.

FIG. 7 illustrates an example environment illustrating ctDNA, which can be utilized to a condition of a subject.

FIG. 8 illustrates an example process for identifying a condition of a subject using fragmentomic data.

FIGS. 9A and 9B illustrate example classification accuracy using methods described herein.

FIG. 10 illustrates one or more devices configured to perform various operations described herein.

### DETAILED DESCRIPTION

Various implementations of the present disclosure relate to techniques for predicting health-related conditions, such as a tumor classification, by nucleic acid sequencing data. In various cases, nucleic acid molecules are obtained from a subject having a condition. In some cases, the nucleic acid molecules include DNA fragments obtained from a liquid biopsy sample. Sequence read data is generated by sequencing the nucleic acid molecules. In various cases, the sequence read data includes at least one dimension that represents a position of the sequenced nucleic acid molecules in a reference genome (also referred to as a “genomic position”), such that the sequence read data is in a spatial domain.

In various implementations of the present disclosure, the sequence read data is preprocessed. In some examples, the sequence read data is preprocessed in the spatial domain. The sequence read data is, in various cases, indicative of endpoint positions of DNA fragments in the sample. According to some examples, the sequence read data is normalized and/or smoothed. In various instances, the sequence read data is scaled based on comparing the sequence read data to baseline sequence read data corresponding to samples associated with the absence of the condition (e.g., there is no detectable presence of the condition in the samples). In some examples, at least one genomic region related to the condition is identified by comparing the baseline sequence read data to benchmark sequence read data. The benchmark sequence read data, in various instances, corresponds to samples associated with the presence of the condition.

In some examples, the sequence read data is transformed into an alternate domain, before or after preprocessing. For instance, the sequence read data may be transformed into a frequency domain or wavelet domain by performing an appropriate transform on the sequence read data. The transformed sequence read data (also referred to as “transformed data”) exhibits various features of the subject that are difficult to impossible to ascertain in the original domain of the sequence read data.

Preprocessing the sequence read data and/or identifying the at least one genomic region related to the condition may improve the efficiency (e.g., reduce the processing time and/or use of computing resources) of analyzing the sequence read data. These features, for instance, are predictive of the condition of the subject. In various examples, features of the preprocessed sequence read data are used to determine the condition of the subject. For instance, the features may be input into a predictive model that is configured to determine whether the subject has the condition. In various cases, indications of the condition of the subject are reported to the subject directly or to a care provider that is responsible for the subject.

Various types of health-related conditions can be predicted using various techniques described herein. In some cases, these techniques are used to determine whether the subject has a cancer type and/or a cancer subtype. For instance, these techniques can be used to determine a genomic subtype of a cancer of the subject. In some examples, these techniques can be used to determine a tumor classification of the subject. For example, these techniques may be used to determine a histological tissue type, a primary site, a tumor dependency, or a tissue origin of a tumor of the subject. In various cases, these techniques can be used to determine whether the subject has a non-cancer condition (e.g., an autoimmune disease).

Implementations of the present disclosure provide significant improvements to the technical field of medical diagnostics and treatment. Utilizing the endpoint data and/or the preprocessing techniques described herein may greatly enhance the accuracy of predictions of health-related conditions based solely on nucleic acid analyses. In some cases, the techniques described herein can be used to predict whether a subject has a particular condition with high (e.g., 85%, 90%, 95%, 99%, or the like) accuracy using nucleic acid molecules that are obtained using a minimally invasive liquid biopsy process. Accordingly, the subject and care providers may make informed decisions about the subject's health without the subject being subjected to highly invasive procedures, such as surgeries (e.g., tissue biopsy procedures). In some examples, the endpoint data and/or the preprocessing techniques described herein may identify new conditions that are not otherwise apparent using previous biomarkers or genomic analyses.

Various analyses described herein cannot be performed in the human mind, or by pen and paper. For example, it would not be possible to preprocess or transform sequence read data representing numerous (e.g., hundreds, thousands, etc.) of bases in a sample into an alternate domain (e.g., a frequency domain) solely in the mind of a human. In addition, it would be impossible to manually or mentally identify relevant features based on the preprocessed sequence read data. Particular implementations of the present disclosure are fundamentally tied to computer technology, and do not represent mere automation of processes that are performed manually or within the human mind.

Implementations of the present disclosure utilize a unique and inventive sample type for predicting occurrence of certain conditions, such as tumor classification and cancer subtype. Previously, tumor classification was identified using histopathological examination of excised tissue or using sequencing-based approaches. Examples of previously used sequencing-based approaches include the detection of specific genomic variants, which may be limited to known regions of interest, and whole genome approaches, which can be limited by resolution and/or depth, using excised tissue. In contrast, the present disclosure describes implementations of predicting conditions using nucleic acid fragments, such as DNA fragments present in blood, plasma, or some other sample type that can be obtained using a minimally invasive procedure. Further, the present disclosure describes implementations of identifying regions of interest associated with conditions, rather than relying solely on known regions of interest. Further, in various implementations described herein, occurrence of certain conditions can be predicted as part of a screening procedure, such as before symptoms develop.

### Example Definitions

As used herein, the terms “deoxyribonucleic acid,” “DNA,” “DNA molecule,” and their equivalents, may refer to a polymer of nucleotides (also referred to as “nucleobases”) containing deoxyribose. The nucleotides in DNA include cytosine (C), guanine (G), adenine (A), and thymine (T). Each DNA nucleotide includes a deoxyribose and a phosphate group. An example single-stranded DNA (ssDNA) molecule includes a chain of covalently bonded DNA nucleotides. In the example ssDNA molecule, the phosphate group of the mth nucleotide is covalently bonded to the deoxyribose of the (m−1)th nucleotide, wherein m is a positive integer greater than 2 and less than or equal to the number of DNA nucleotides in the chain. In various examples, DNA is double-stranded and includes two ssDNA molecules that are complementary to one another and coiled around each other in a double helix form. The nucleotides of one ssDNA molecule are hydrogen bonded to the nucleotides of the other ssDNA molecule. In particular, the pyrimidines (A and T) hydrogen bond to each other, and the purines (C and G) hydrogen bond to each other.

As used herein, the terms “ribonucleic acid,” “RNA,” “RNA molecule,” and their equivalents, may refer to a polymer of nucleotides containing ribose. The nucleotides in RNA include cytosine (C), guanine (G), adenine (A), and uracil (U). Each RNA nucleotide includes a ribose and a phosphate group. In an example RNA molecule, the phosphate group of the nth nucleotide is covalently bonded to the ribose of the (n−1)th nucleotide, wherein n is a positive integer greater than 2 and less than or equal to the number of RNA nucleotides in the chain. Messenger RNA (mRNA) is a type of RNA molecule that is synthesized (or “transcribed”) by RNA polymerase (an enzyme) to be complementary to a gene encoded in a DNA sequence, and is also used by a ribosome to synthesize a polypeptide or protein. An mRNA is therefore an example of a “coding RNA.” In various cases, intron sequences are removed from an mRNA via a process known as “RNA splicing.” MicroRNA (“miRNA”) are single-stranded RNA molecules that perform post-transcriptional gene expression regulation. For instance, a miRNA may bind to a complementary mRNA molecule, thereby cleaving, destabilizing, or otherwise preventing the mRNA molecule from being translated into a polypeptide or protein by a ribosome. In various examples, a miRNA has a length in a range of 21 to 23 RNA nucleotides. As used herein, the terms “non-coding RNA” may refer to a type of RNA that is not translated into a protein. Examples of non-coding RNA include miRNA, transfer RNA (tRNA), and ribosomal RNA (rRNA). The term “functional RNA,” and its equivalents, may refer to any RNA molecule that impacts a biological process. For instance, functional RNA may include mRNA, miRNA, tRNA, rRNA, and the like.

As used herein, the term “base,” and its equivalents, may refer to a monomer of a polymer. For example, a base of DNA or RNA is a nucleotide.

As used herein, the term “base pair,” and its equivalents, may refer to a pair of complementary DNA nucleotides, which are hydrogen-bonded to one another in a double-stranded DNA molecule. For example, a base pair includes a first base in a first ssDNA and a second base in a second ssDNA, wherein the first and second bases are complementary and hydrogen-bonded to one another.

As used herein, the terms “nucleotide,” “nucleobase,” “nucleic acid,”“ ” “nucleic acid molecule,” and their equivalents, may refer to an organic molecule that includes a nitrogenous base, a sugar, and a phosphate group. In various cases, a nucleotide is a monomer of DNA or RNA. A nucleotide, for instance, is a chemical structure.

As used herein, the terms “3′ end,” “3-prime end,” and their equivalents, may refer to a terminus of a single-stranded nucleotide polymer that includes a base whose third carbon in its deoxyribose or ribose is bound to a hydroxyl group while being unbound to another base.

As used herein, the terms “5′ end,” “5-prime end,” and their equivalents, may refer to a terminus of a single-stranded nucleotide polymer that includes a base whose fifth carbon in its deoxyribose or ribose ring is unbound to another base. In some cases, the fifth carbon is bound to a phosphate group.

As used herein, the “length” of a polymer refers to a number of covalently bonded monomers that are included in the polymer. For instance, the length of a DNA molecule may be the number of covalently bonded nucleotides in at least one strand of the DNA molecule and/or the number of base pairs in the DNA molecule. In various examples, the length of an RNA molecule may be the number of covalently bonded nucleotides in the RNA molecule.

As used herein, the term “gene,” and its equivalents, refers to a sequence of DNA nucleotides that is transcribed into a functional RNA. The functional RNA, for instance, is RNA that is translated into a polypeptide or protein (e.g., mRNA) or that has some other biological function (e.g., miRNA, tRNA, etc.). A gene is “expressed” when it is used as a template to generate a functional RNA. A subject, for instance, has numerous genes contained in the subject's genome. A gene may include both introns and exons. As used herein, the term “intron,” and its equivalents, may refer to a subset of DNA nucleotides in a gene that is not used to code for any functional RNA that is expressed by the organism. As used herein, the term “exon,” and its equivalents, may refer to a subset of DNA nucleotides in a gene that is used to code for a functional RNA. For instance, an exon may encode a polypeptide or protein that is expressed by the organism. In various examples, a gene can be represented in data (e.g., as data representative of the sequence of DNA nucleotides in the gene) or as a chemical structure (e.g., as the sequence of DNA nucleotides itself).

As used herein, the term “genome,” and its equivalents, refers to the aggregate of genes of a subject. In various cases, a genome represents the sequences of several linear DNA molecules that are present in a subject's chromosomes. A “reference genome” refers to an aggregation of genes of one or more reference subjects. In various cases, a genome is represented in data.

As used herein, the terms “pangenome,” “pan-genome,” “supragenome,” and their equivalents, refers to an aggregate set of genes from multiple subgroups (e.g., strains) within a population (e.g., a clade) of subjects. A pangenome, for example, indicates genes that are present in all subjects within the population, as well as genes that are present in some of the subjects of the population. A pangenome is represented in data, for instance.

As used herein, the term “transcriptome,” and its equivalents, refers to the aggregate of RNA sequences of a subject. In some cases, a transcriptome is limited to mRNA sequences. In various examples, a transcriptome is represented in data.

As used herein, the term “genomic DNA,” “gDNA,” “chromosomal DNA,” and their equivalents, may refer to DNA molecules that are obtained from a chromosome and/or nucleus of a cell.

As used herein, the terms “DNA fragment,” “fragment,” and their equivalents, may refer to DNA molecules that are excised and/or broken off from a larger DNA molecule.

As used herein, the terms “cell-free DNA,” “cfDNA,” and their equivalents, may refer to DNA fragments that are non-encapsulated and obtained outside of cells within a sample (e.g., a liquid biopsy sample).

As used herein, the terms “circulating tumor DNA,” “ctDNA,” and their equivalents, may refer to a cfDNA molecule that originates from a cancer cell.

As used herein, the term “promoter,” and its equivalents, may refer to a portion of a DNA molecule that binds one or more proteins in order to initiate transcription of a gene. For example, the promotor is located “upstream” of the gene. For example, the promotor is located between the 5′ end of the DNA molecule and the gene. A promotor may include one or more binding sites for RNA polymerase, and/or one or more transcription factor binding sites. In some examples, a promotor includes one or more CpG islands. A promoter, for instance, includes a transcription start site.

As used herein, the terms “CpG island,” “CGI,” “CpG site,” and their equivalents, may refer to a continuous portion of a DNA molecule whose sequence includes greater than a threshold amount (e.g., greater than 50%) of G-C base pairs.

As used herein, the term “enhancer,” and its equivalents, may refer to a portion of a DNA molecule that binds one or more proteins in order to increase the chance that a gene will be transcribed. For instance, an enhancer includes one or more transcription factor binding sites. In various cases, an enhancer includes one or more CpG islands.

As used herein, the term “cancer,” and its equivalents, may refer to a condition of a subject in which particular cells (referred to as “cancer cells”) divide uncontrollably in the subject's body. In some cases, a cancer is characterized by a location or tissue type from which the cancer cells originated. In some examples, a cancer is characterized by a location or tissue type in which the cancer cells are located.

As used herein, the terms “tumor,” “neoplasm,” and their equivalents, may refer to a mass of tissue including cancer cells.

As used herein, the terms “tissue of origin,” “tissue origin,” and their equivalents, refers to a differentiated type of tissue from which cancer cells in the body of a subject began dividing uncontrollably in the subject's body.

As used herein, the terms “liquid biopsy,” “fluid biopsy,” and their equivalents, may refer to a process of obtaining a fluid sample from a subject's body. The sample, for instance, can be referred to as a “liquid biopsy sample.” Examples of fluids that are sampled from the body include blood, plasma, cerebrospinal fluid, sputum, stool, urine, lymphatic fluid, and saliva.

As used herein, the term “tissue biopsy,” and its equivalents, may refer to a process of obtaining a sample of cells from a subject's body. A tissue biopsy, in various cases, is performed by cutting a mass of cells from the subject's body. For instance, a tissue biopsy is a procedure performed by a surgeon, interventional radiologist, interventional cardiologist, or other specialized clinician. The term “tissue” or “tissue biopsy sample” can be used to refer to the sample of cells obtained using a tissue biopsy.

As used herein, the term “subject,” and its equivalents, may refer to a human or non-human animal. A subject that is receiving care from at least one care provider may be referred to as a “patient.” As used herein, the terms “machine learning,” “ML,” “computer learning,” “artificial intelligence,” and their equivalents, may refer to the use of a computing devices to learn patterns in training data. The process of learning these patterns may be referred to as “training.” In particular cases, one or more computing devices may perform machine learning by executing a machine learning model. As used herein, the terms “machine learning model,” “ML model,” and their equivalents, may refer to data encoding instructions that, when executed by at least one computing device, causes the at least one computing device to learn patterns in training data by optimizing one or more metrics, values, or other types of parameters. After training, an ML model, when executed by at least one computing device, causes the at least one computing device to utilize the optimized parameters in order to perform one or more tasks.

As used herein, the term “variant,” and its equivalents, may refer to a difference between a subject genetic sequence and a reference sequence. For instance, a variant may correspond to a difference between one or more nucleotides in a genome of a subject and one or more corresponding nucleotides in at least one reference genome or pangenome. A variant may be characterized by its identity (e.g., what nucleotides are different), its position (e.g., where are the nucleotides located in the genome, what chromosome contains the nucleotides, what gene contains the nucleotides, etc.), its length (e.g., how many nucleotides are different from the reference sequence), its type (e.g., substitution, insertion, deletion, copy number alternation, rearrangement of fusion, etc.), and other features that indicates its significance and/or relevance. In some cases, a variant represents any apparent alteration in a sequence that has been read from a nucleic acid molecule with respect to the reference sequence, such as reads cleaved by restriction enzymes (RE). In various examples, a variant can be represented in data (e.g., by data characterizing the variant) or as a chemical structure (e.g., the nucleotides themselves). As used herein, the term “mutation,” and its equivalents, may refer to a change in a gene.

As used herein, the term “substitution,” and its equivalents, can refer to a nucleotide in a subject sequence that is different than an equivalent nucleotide (e.g., a nucleotide at the same position) in a reference sequence.

As used herein, the term “insertion,” and its equivalents, can refer to a nucleotide in a subject sequence that is added with respect to a reference sequence.

As used herein, the term “deletion,” and its equivalents, can refer to the removal of a nucleotide from a nucleotide sequence.

As used herein, the terms “copy number alternation,” “CNA,” “copy number variation,” “CNV,” and their equivalents, can refer to a portion of a reference sequence that is repeated.

As used herein, the terms “rearrangement of fusion,” “fusion rearrangement,” “translocation,” and their equivalents, can refer to a change in the relative position of one or more portions of a reference sequence, thereby generating a gene that was not present in the reference sequence.

As used herein, the term “sequencing,” and its equivalents, may refer to a process of identifying the order and identity of monomers in a polymer chain, such as the order and identity of nucleotides in a DNA or RNA molecule. The terms “whole genome sequencing,” “WGS,” and their equivalents, may refer to the process of sequencing an entire genome of a subject, including the introns and exons of the genes of the subject. The term “whole exome sequencing,” and its equivalents, may refer to the process of sequencing all exomes of a subject. The term “targeted sequencing,” and its equivalents, may refer to the process of sequencing a portion of the genome of a subject, such as sequencing a single gene of the subject. Various techniques can be utilized to sequence a DNA or RNA molecule, such as massively parallel sequencing (MPS), nanopore sequencing, direct sequencing, Sanger sequencing, or next-generation sequencing. In various cases, sequencing is performed on physical molecules (e.g., RNA or DNA) and is used to generate data.

As used herein, the terms “massive parallel sequencing,” “massively parallel sequencing,” “MPS,” and their equivalents, may refer to a technique for simultaneously performing multiple reactions that can be used to identify the order and identity of monomers in multiple polymer chains. In particular cases, massive parallel sequencing can be performed using sequencing-by-synthesis on clonally amplified DNA molecules that are located in spatially separated regions, which are individually monitored by sensors.

As used herein, the term “nanopore sequencing,” and its equivalents, may refer to a technique for identifying the order and identity of monomers in a polymer chain by transporting the polymer chain from a first space to a second space, wherein the first space and the second space are separated by a substrate, by directing the polymer chain through a small hole (known as a “nanopore”) embedded in the substrate, and monitoring a relative electrical signal (e.g., a voltage or current) between the first space and the second space.

As used herein, the term “sensor,” and its equivalents, may refer to a physical device or other apparatus that is configured to detect one or more detection signals.

As used herein, the term “detection signal,” and its equivalents, may refer to a physical signal that can be identified, characterized, or otherwise perceived by a sensor.

As used herein, the term “sequence read data,” and its equivalents, may refer to data that is indicative of an order and identity of monomers in a polymer, such as the order and identity of nucleotides in a DNA or RNA sequence. In various implementations, sequence read data is generated via a sequencing operation.

As used herein, the term “image,” and its equivalents, may refer to 2D or 3D array of data indicative of an array of pixels or voxels.

As used herein, the term “ligating,” and its equivalents, may refer to a process of joining two molecules together, for example, with a chemical bond.

As used herein, the term “adapter,” and its equivalents, may refer to an oligonucleotide that can be ligated to a target nucleic acid molecule. In various cases, an adapter prepares the target nucleic acid molecule for sequencing.

As used herein, the term “bait molecule,” and its equivalents, may refer to a nucleic acid molecule having a region that is complementary to a region of a target molecule (e.g., cfDNA). A bait molecule includes, for instance, a nucleic acid molecule that can hybridize to (i.e., is complementary to) a target molecule can be used to capture the target molecule. In some instances, the bait molecule is a capture oligonucleotide (or capture probe). In some instances, the bait molecule is suitable for solution phase hybridization to the target molecule. In some instances, the bait molecule is suitable for solid phase hybridization to the target molecule. In some instances, the bait molecule is suitable for both solution-phase and solid-phase hybridization to the target molecule. The design and construction of bait molecules is described in more detail in, e.g., International Patent Application Publication No. WO 2020/236941.

As used herein, the term “amplifying,” and its equivalents, may refer to a process of generating copies of a target molecule, such as a nucleic acid molecule.

As used herein, the term “hybridization,” and its equivalents, may refer to a process by which to complementary single-stranded nucleic acid molecules bind to one another, thereby forming a double-stranded nucleic acid molecule. In certain examples, the double-stranded nature of the nucleic acid molecule is maintained under stringent hybridization conditions. Exemplary stringent hybridization conditions include an overnight incubation at 42° C. in a solution including 50% formamide, 5XSSC (750 mM NaCl, 75 mM trisodium citrate), 50 mM sodium phosphate (pH 7.6), 5XDenhardt's solution, 10% dextran sulfate, and 20 μg/ml denatured, sheared salmon sperm DNA, followed by washing the filters in 0.1XSSC at 50° C.

As used herein, the term “complementary,” and its equivalents, may refer to a state of two single-stranded nucleic acid molecules with respective sequences that cause the nucleic acid molecules to spontaneously hybridize to one another. One nucleic acid molecule, for instance, may have a sequence that causes each nucleic acid to hydrogen bond to a respective nucleic acid in the other nucleic acid molecule.

As used herein, the terms “therapy,” “treatment,” and their equivalents, may refer to a composition or process that can be used to remediate a health problem. Cancer therapies, for instance, include surgery, radiotherapy, chemotherapy, immunotherapy, cell-based therapies, and the like. Examples of cancer therapies include abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), Lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximabcmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecanhziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), and combinations thereof. Examples of cancer therapies also include targeted antibody-based therapies (antibody-drug conjugates, antibody-radioisotope conjugates, and targeted immune cell therapies (e.g., immune effector cells genetically modified to express a chimeric antigen receptor (CAR).

As used herein, the term “treatment-responsive,” and its equivalents, may refer to a type of cancer cells that can be substantially killed, or prevented from dividing, using a predetermined type of therapy. For example, cancer cells of a subject may be responsive to a particular treatment if, after the subject is administered the treatment, the cancer cells are diminished by a particular progression level (e.g., radiographic progression level, marker-based progression level, such as prostate-specific antigen (PSA) progression, etc.). Accordingly, the responsiveness of the cells to the type of therapy may indicate the effectiveness of that therapy.

As used herein, the term “treatment-resistant,” and its equivalents, may refer to a type of cancer that cannot be substantially killed using a predetermined type of therapy.

As used herein, the term “metastasis profile,” and its equivalents, may refer to a propensity of a type of cancer to metastasize into one or more differentiated tumor types besides the cancer's tissue origin. In some implementations, the metastasis profile can further indicate the type of tissue in which the cancer can or is likely to metastasize.

As used herein, the term “clinical trial,” and its equivalents, may refer to a research study used to evaluate a hypothesis based on participation by one or more subjects. In various examples, a clinical trial can be used to assess the efficacy and/or safety of a proposed therapy. A clinical trial may be performed in furtherance of approval of a treatment by a regulatory authority (e.g., the United States Food & Drug Administration (FDA)).

### Description of Example Implementations

Various implementations of the present disclosure will now be described with reference to the accompanying Figures.

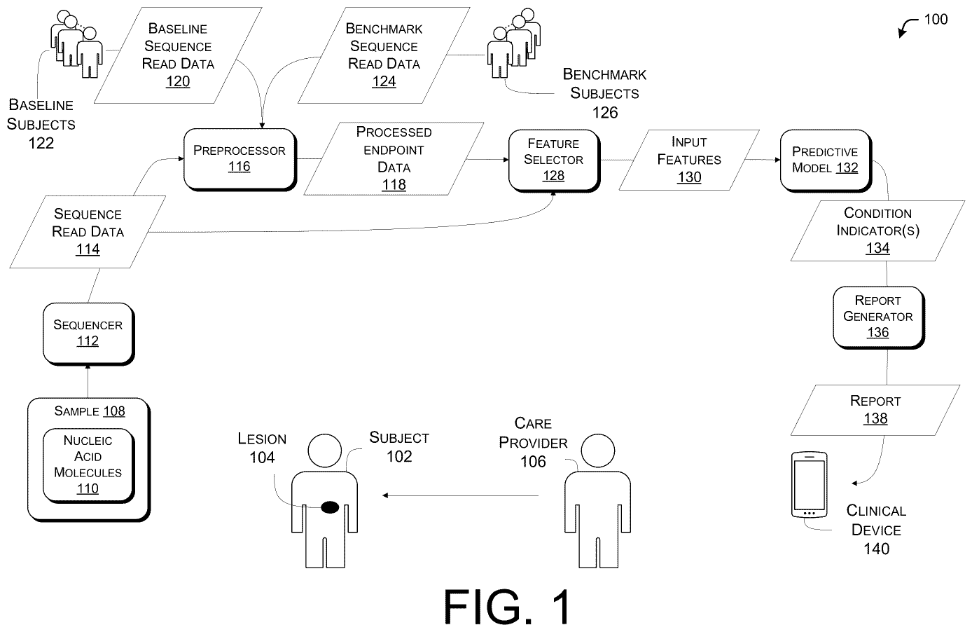

FIG. 1 illustrates an example environment 100 for predicting a condition of a subject 102 based on fragmentomic features of the subject 102. In some cases, the subject 102 lacks any apparent disease or other pathological condition. For example, the subject 102 may present to a clinical environment for an assessment of a condition of the body of the subject 102, such as the general health or well-being of the subject 102. In various cases, the subject 102 presents to the environment 100 as part of a screening assessment for the condition. For instance, the subject 102 may schedule an appointment in the environment 100 based on an age, demographic, or a family history of the condition of the subject 102, rather than in response to any symptom or suspected condition.

In various implementations, the subject 102 has a disease or a suspected disease. The subject 102, for instance, may present to the clinical environment with a lesion 104. In various cases, the lesion 104 may be a tumor that includes cancer cells. According to various examples, the subject 102 has one or more types of cancer, such as adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.

In some embodiments, the subject 102 has a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms'tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

While FIG. 1 illustrates the subject 102 having a lesion 104, implementations of the present disclosure are not so limited. In various implementations, the subject 102 may have a non-cancer condition. For instance, the subject 102 may have a genetic disorder, diabetes, cardiac disease, a respiratory disease, an infectious disease, an autoimmune disease, or another pathological condition.

In various cases, a care provider 106 (also referred to as a “healthcare provider”) is responsible for diagnosing and/or treating the subject 102. According to some implementations, the condition of the subject may be initially identified using a noninvasive technique. For example, the lesion 104 may be visualized using an imaging modality, such as ultrasound, x-ray, computed tomography (CT), magnetic resonance imaging (MRI), positron emission tomography (PET), single photon emission CT (SPECT), or any combination thereof. Using the noninvasive technique, the care provider 106 may identify the presence of the lesion 104 but may be unable to determine whether the lesion 104 is a cancerous tumor using noninvasive diagnostic methodologies. In some cases in which the lesion 104 is a tumor, the care provider 106 may be unable to identify whether the tumor is metastatic or benign, or may be unable to otherwise categorize the tumor.

In some examples, the care provider 106 may be unable to identify a characteristic of a subject presenting with a disease based on the noninvasive technique, wherein the characteristic is determinative of, or at least correlated with, an effectiveness of at least one therapy at treating the disease, an ineffectiveness of at least one therapy at treating the disease, a survivability (e.g., a likelihood that the subject will survive by a predetermined date or time), an expected quality of life, at least one predetermined symptom, at least one comorbidity, another factor relevant to the prognosis associated with the disease, or any combination thereof.

In some examples, the care provider 106 could identify a condition (e.g., cancer) of the subject 102 using histochemistry and/or immunohistochemistry. For instance, the care provider 106 could surgically remove a tissue sample from the lesion 104 and/or review the tissue sample using histochemistry and/or immunohistochemistry. However, attempting to classify the lesion 104 using these techniques has several drawbacks. First, the tissue sample may not be classifiable using conventional histological techniques, such as conventional immunohistochemical staining and review. Second, it is unlikely that the single care provider 106 would be trained to perform the tissue biopsy (which would be performed by a surgeon), to administer anesthesia to the subject 102 during the tissue biopsy (which would be performed by an anesthesiologist), and the analysis of the tissue biopsy (which would be performed by a trained pathologist), such that the classification would utilize multiple highly trained care providers. Even if the lesion 104 was classifiable by these means, the coordinated efforts of these care providers could delay classification of the lesion 104 and could cause significant expense to the subject 102. In various examples, the delay in classification could cause significant emotional hardship to the subject 102, who could be prevented from receiving an informed prognosis for weeks. Further, the delay in classification could delay administration of a therapy to the subject 102 in order to treat the lesion 104, which could cause lasting harm to the subject 102, particularly in cases in which the lesion 104 is representative of an aggressive form of cancer.

In various implementations of the present disclosure, a condition of the subject 102 can be determined without performing histochemistry and/or immunohistochemistry. For instance, a sample 108 is obtained from the subject 102. In some cases, the sample includes a liquid biopsy sample. The liquid biopsy sample 108, for instance, includes blood, plasma, cerebrospinal fluid, sputum, stool, urine, lymphatic fluid, saliva, or some other fluid obtained from the body of the subject 102. In some cases, a blood sample is obtained intravenously from the subject 102. The liquid biopsy sample 108, according to various examples, is a plasma sample obtained from the blood of the subject 102. The liquid biopsy sample 108, for instance, can be obtained in a minimally invasive procedure, which could be performed by a medical technician rather than a surgeon. In some examples, the sample 108 includes a tissue biopsy sample. For instance, the sample 108 is obtained by removing cells from the lesion 104 and from the subject 102. In some cases, the tissue biopsy sample is surgically excised from the subject 102.

The sample 108 includes nucleic acid molecules 110. According to some examples, the nucleic acid molecules 110 include genomic DNA (gDNA). For instance, the nucleic acid molecules 110 include chromosomal DNA that is located in, or extracted from, cells in the sample 108. According to some cases, the DNA is extracted from nuclei and the cells in the sample 108 using mechanical shearing and/or the introduction of a chemical (e.g., a detergent). The DNA may be subsequently isolated from proteins and other cellular materials. In some implementations, the nucleic acid molecules 110 indicate an entire genome of the subject 102 and/or the lesion 104. Thus, a genome of the subject 102 and/or the lesion 104 can be determined by sequencing the DNA in the nucleic acid molecules 110.

In some examples, the nucleic acid molecules 110 include RNA. In some implementations, the nucleic acid molecules 110 include messenger RNA (mRNA), microRNA, non-coding RNA, functional RNA, or any combination thereof. Various RNA in the nucleic acid molecules 110 may be indicative of proteins expressed in the cells of the subject 102 and/or the lesion 104.

In some cases, the sample 108 includes cell-free DNA (cfDNA). In examples in which the subject 102 has cancer (e.g., the lesion 104 is a cancerous tumor), the cfDNA, for instance, includes circulating tumor DNA (ctDNA) and/or non-ctDNA. In cases wherein the lesion 104 is a tumor, cancer cells within the lesion 104 will lyse and release the ctDNA into the bloodstream of the subject 102. These cancer cells, for example, include circulating tumor cells (CTCs). Further, other cells additionally release non-ctDNA into the bloodstream of the subject. In general, the cfDNA includes fragments with lengths that are in a range of 1 to 500, 3 to 500, or 100 to 500 bases long. For instance, the cfDNA includes fragments that are about 170 bases long and/or fragments that are about 340 bases long. For example, the cfDNA includes fragments that are 100 to 240 bases long and/or fragments that are 270 to 410 bases long.

In various cases, the sample 108 is transported to a location that is remote from the subject 102 for further processing. For example, the sample 108 is removed from the subject 102 in a clinical environment (e.g., a hospital) and is then transported to a remote laboratory for further testing and analysis.

A sequencer 112 is configured to generate sequence read data 114 indicating the sequences of the nucleic acid molecules 110. The sequencer 112, for instance, includes one or more devices that are configured to generate the sequence read data 114 by processing at least a portion of the sample 108. In some cases, the nucleic acid molecules 110 are extracted from the sample 108. The extraction can be performed by the sequencer 112, by another device, manually (e.g., by a laboratory technician), or any combination thereof. Any appropriate extraction method known to those of ordinary skill in the art can be utilized.

In various cases, the sequencer 112 is configured to perform one or more processes (e.g., chemical reactions) on the nucleic acid molecules 110 in order to prepare the nucleic acid molecules 110 for sequencing. For instance, the sequencer 112 may ligate adapters onto the nucleic acid molecules 110 and/or amplify the nucleic acid molecules 110, such that numerous copies of the ligated nucleic acid molecules 110 are available for sequencing. Examples of the adapters include, for example, amplification primers, flow cell adapter sequences, substrate adapter sequences, or sample index sequences. The nucleic acid molecules 110 (e.g., the ligated nucleic acid molecules 110) may be amplified by generating multiple copies of the nucleic acid molecules 110 using one or more techniques such as polymerase chain reaction (PCR), a non-PCR amplification technique, or an isothermal amplification technique.

The sequencer 112 may identify the length, position, and identity of the bases in the nucleic acid molecules 110 by sequencing the nucleic acid molecules 110 (e.g., the amplified and/or ligated nucleic acid molecules 110). In various cases, the sequencer 112 is a next-generation sequencer configured to perform next-generation sequencing (NGS) on the nucleic acid molecules 110. In various implementations, the sequencer 112 utilizes first-generation sequencing (e.g., Sanger sequencing), second-generation sequencing (e.g., massive parallel sequencing), third-generation sequencing (e.g., nanopore sequencing), or a combination thereof. In some cases, the sequencer 112 is configured to sequence substantially all of the nucleotides of all of the nucleic acid molecules 110 fragments obtained from the sample 108. In some examples, the sequencer 112 is configured to perform targeted sequencing. For instance, the sequencer 112 may determine whether the nucleic acid molecules 110 fragments contain one or more predetermined sequences at one or more genomic locations.

In various cases, the sequencer 112 includes one or more sensors that are configured to detect physical signals (also referred to as “detection signals”) that are indicative of the nucleotide sequences of the nucleic acid molecules 110. The sequencer 112 may perform sequencing-by-synthesis. For example, the sequencer 112 may include one or more optical sensors configured to detect optical signals emitted from fluorescently tagged nucleotide triphosphates (NTPs) that are joined together in a synthesized DNA strand using the ligated nucleic acid molecules 110 as templates. The optical signals detected by the optical sensor(s), for instance, are indicative of the sequences of the nucleic acid molecules 110. The sequencer 112 may perform nanopore sequencing. In various cases, the sequencer 112 includes one or more electrical sensors configured to measure an electrical signal (e.g., an electrical current) across a substrate as the ligated nucleic acid molecules 110 are directed through a nanopore extending through the substrate. The electrical signal over time, in various cases, is indicative of the sequences of the nucleic acid molecules 110 in the sample 108. The sequencer 112, in various implementations, is configured to generate the sequence read data 114 as digital data based on the analog signals detected by the sensor(s). For instance, the sequencer 112 includes one or more analog to digital converters (ADCs). In various cases, the sequencer 112 includes at least one processor configured to generate the sequence read data 114.

In some implementations, the sequencer 112 performs RNA sequencing (RNA-seq) on the nucleic acid molecules 110. For example, the nucleic acid molecules 110 include RNA that is extracted from the sample 108. In some examples, the RNA in the nucleic acid molecules 110 is fragmented. In various implementations, complementary DNA (cDNA) is generated using reverse transcriptase, such that the cDNA includes sequences that are complementary to the RNA in the nucleic acid molecules 110 from the sample 108. The cDNA, according to various cases, can be sequenced using the DNA sequencing techniques described above. Accordingly, in some cases, the sequence read data 114 indicates sequences of RNA present in the sample 108, which may be indicative of the transcriptome of the subject 102 and/or the lesion 104.

In various cases, the sequencer 112 performs sequencing on a subset of the nucleic acid molecules 110. For instance, the sequencer 112 may perform targeted sequencing on portions of the nucleic acid molecules 110 that correspond to one or more predetermined genes, such as any of the specific genes described herein. Other portions of the genome may be specifically sequenced, such as promoters, hotspots, CpG sites, or other portions of the genome that are not specifically genes but have an impact on genomic expression. The sequencer 112, in some cases, may refrain from sequencing at least a portion of the nucleic acid molecules 110 that do not correspond to the subset.

The sequence read data 114, according to various instances, is in a spatial domain. For example, the sequence read data 114 may be indicative of the genomic locations of DNA fragments among the nucleic acid molecules 110 in the sample 108. The sequence read data, in some examples, is aligned with at least one reference sequence (e.g., a reference genome). Accordingly, the bases of nucleic acid molecules 110, for instance, correspond to genomic positions with respect to the reference sequence(s).

The sequence read data 114, in various implementations, is indicative of endpoints of the nucleic acid molecules 110 (referred to herein as “endpoint data”). Endpoint data may include endpoint positions, including left endpoint positions and/or right endpoint positions. “Endpoint positions,” as used herein, refers to the two bounds of the range of genomic positions associated with a nucleic acid molecule. The two endpoints may be referred to as a “start endpoint” and an “end endpoint,” or as a “left endpoint” and a “right endpoint.” Endpoint data may include a length of the nucleic acid molecules 110. In various examples, the endpoint data may be difficult to analyze directly. For instance, although it may be possible to identify, using the endpoint data, attributes or other characteristics that are predictive of the condition of the subject 102, such analyses may utilize numerous processing resources.

In some examples, the sequence read data 114 and/or the endpoint data is preprocessed by a preprocessor 116 to generate processed endpoint data 118. According to various implementations, features of the sequence read data 114 indicative of the condition of the subject 102 may be difficult to ascertain from the sequence read data 114 directly. In some cases, the features of the sequence read data 114 indicative of the condition of the subject 102 can be identified more efficiently by analyzing the processed endpoint data 118. Accordingly, generating the processed endpoint data 118, in various examples, can greatly reduce the amount of processing resources utilized to identify the condition of the subject 102. Further, in some cases, generating the processed endpoint data 118 enables new characteristics to be identified using the sequence read data 114.

In various implementations, the processed endpoint data 118 may include a visual representation of the endpoint counts indicated by the nucleic acid molecules 110 across at least one genomic region. In some cases, the processed endpoint data 118 may include a two-dimensional and/or a three-dimensional representation of the endpoint data. In various instances, the processed endpoint data 118 includes a one-dimensional representation of the endpoint counts indicated by the nucleic acid molecules 110 across at least one genomic region. For instance, the processed endpoint data 118 may include an array or the like. In some cases, the at least one genomic region is continuous. In some cases, the at least one genomic region is non-contiguous.

The preprocessor 116 may generate the endpoint data by analyzing the endpoint counts indicated by the nucleic acid molecules 110. For instance, the preprocessor 116 may determine a number of endpoint positions at each genomic position in one or more genomic regions based on analyzing the sequence read data 114. In some examples, the preprocessor 116 determines the left endpoint counts and/or the right endpoint counts of the nucleic acid molecules.

In various implementations, the preprocessor 116 uses one or more techniques to generate the processed endpoint data 118 based on the endpoint data. The preprocessor 116, in some examples, is configured to normalize the endpoint data. Global (e.g., whole genome) coverage differences may arise due to experimental and/or environmental factors, such as amplification bias, sample degradation, or the like. Certain genomic regions may have a higher sequencing rate, for instance, due to the sequencing technique utilized to generate the sequence read data 114. In some examples, a genomic region may have higher endpoint counts due to amplification of the genomic region, or alternatively lower endpoint counts due to, for instance, gene deletion. Normalizing the endpoint data can control for sample-to-sample variation, copy number variation, or sampling artifacts that arise due to the sequencing technique utilized. For example, the preprocessor 116 may normalize the endpoint counts at a particular genomic position to a mean of the endpoint counts across one or more genomic regions. In various cases, the preprocessor 116 normalizes the endpoint data with respect to another metric (e.g., a median, a minimum, a maximum, a standard deviation, or the like) of the endpoint data. In some examples, the preprocessor 116 normalizes endpoint counts within a particular genomic locus to a metric (e.g., a mean) of the particular genomic locus. In some cases, the endpoint counts may be normalized to a ratio of the ctDNA to the cfDNA in the sample 108. In some examples, the endpoint counts may be first normalized within a particular genomic locus, and then normalized to a ratio of the ctDNA to the cfDNA in the sample 108. In some examples, the endpoint counts normalized to a ratio of the ctDNA to the cfDNA in the sample 108 after smoothing and/or scaling the endpoint data.

In various examples, the preprocessor 116 is configured to smooth the endpoint data. For example, the preprocessor 116 may generate a metric over a window of genomic positions centered on a particular genomic position. The metric may include a mean endpoint count, a weighted mean endpoint count, a median endpoint count, a kernel function, a filter or the like. Examples of kernel functions include a linear, a polynomial, a Gaussian, an exponential, or a Laplacian kernel function. Examples of filters include a Butterworth filter, a Chebyshev filter, a finite impulse response (FIR) filter, or an infinite impulse response (IIR) filter. In some cases, the filter applied by the preprocessor 116 is a low-pass filter, a high-pass filter, or a bandpass filter. For instance, the filter may be defined by one or more cutoff frequencies. The window of genomic positions may be in a range of 1 to 100 genomic positions. In various cases, the preprocessor 116 assigns, to the particular genomic position, the metric corresponding to the window of genomic positions centered on the particular genomic position. For instance, the preprocessor 116 may assign the metric corresponding to the window to the particular genomic position. In various cases, the preprocessor may perform one or more local regression analyses to determine the metric. In some examples, the preprocessor 116 may determine a local fit (e.g., a linear fit, a quadratic fit, a polynomial fit, or the like) for one or more genomic regions. The preprocessor 116 may assign the value of the local fit to each genomic position in the one or more genomic regions.

In some implementations, the preprocessor 116 is configured to scale the endpoint data. For instance, the preprocessor 116 may identify baseline sequence read data 120 corresponding to baseline subjects 122. The baseline sequence read data 120 is, in some examples, indicative of baseline nucleic acid molecules in samples collected from the baseline subjects 122. The baseline sequence read data 120, in various instances, is indicative of baseline endpoint counts of the baseline nucleic acid molecules with respect to reference sequence(s) (e.g., a reference genome). In some implementations, the endpoint counts and the control endpoint counts are determined with respect to the same reference sequence(s). In some examples, the baseline subjects 122 include subjects without the condition. The baseline subjects 122, in various cases, include subjects with low-shedding tumors. For example, baseline samples collected from the baseline subjects 122 are associated with an absence of ctDNA. In various examples, the baseline samples are determined to be free of tumors based on having a ctDNA tumor fraction estimate of zero. In various examples, the baseline subjects 122 have a predetermined subtype of the condition. In some implementations, the baseline subjects 122 include subjects who do not have cancer.

In some examples, the preprocessor 116 identifies or generates baseline endpoint data based on the baseline sequence read data 120. The baseline endpoint data may be normalized and/or smoothed. In various cases, the preprocessor 116 may generate baseline distance metrics that are indicative of the difference between the endpoint data and the baseline endpoint data. The baseline distance metrics can be utilized to identify genomic regions associated with the condition. For instance, the baseline distance metrics may be indicative of a statistical significance between normal samples (e.g., samples from individuals who do not have the condition) and abnormal samples (e.g., samples from individuals who have the condition). The baseline distance metrics, in some examples, are in a z-score space. For instance, the preprocessor 116 may determine a difference between a value of the endpoint data and the mean of the baseline endpoint data at a genomic position. The difference between the value of the endpoint data and the mean of the baseline endpoint data is, in some cases, divided by a standard deviation of the baseline endpoint data at the genomic position to determine the z-score. The value at a genomic position, in various instances, is replaced with the corresponding z-score for the genomic position to scale the endpoint data.

In various implementations of the present disclosure, the sequence read data 114 and/or the processed endpoint data 118 is output to a data transformer rather than analyzed directly. The data transformer is configured to generate transformed data by transforming the sequence read data 114 from a first domain (e.g., the spatial domain) to a second domain that is different than the first domain. That is, the second domain is an “alternate” domain to the first domain. In some cases, the transformed data includes data representing the sequence read data 114 in the second domain. In some examples, the transformed data includes one or more images representing the sequence read data 114 in the second domain.

Various types of transformations can be performed by the data transformer. In some examples, the data transformer is configured to generate the transformed data by performing a Fourier transform on the sequence read data 114 and/or the processed endpoint data 118. The transformed data, for instance, is in a frequency domain. According to some examples, the data transformer is configured to perform a Fast Fourier Transform (FFT) on the sequence read data 114. In some cases, the data transformer is configured to perform a continuous Fourier transform on a function representative of the sequence read data 114 and/or the processed endpoint data 118. In various examples, the data transformer is configured to perform a discrete Fourier transform (DFT) on the sequence read data 114 and/or the processed endpoint data 118. According to some cases, the data transformer is configured to perform a short-time Fourier transform (STFT) on the sequence read data 114 and/or the processed endpoint data 118.

In some examples, the data transformer is configured to generate the transformed data using one or more other types of transforms. For example, the data transformer may generate the transformed data by performing a Hartley transform, a Laplace transform, a Mellin transform, a wavelet transform (e.g., a continuous wavelet transform (CWT), a discrete wavelet transform (DWT), a fast wavelet transform (FWT), a complex wavelet transform, a Newland transform, a stationary wavelet transform (SWT), a second generation wavelet transform (SGWT), a dual-tree complex wavelet transform (DTCWT), etc.), or any combination thereof, on the sequence read data 114 and/or the processed endpoint data 118. In some cases, the data transformer generates the transformed data by generating a Taylor series or Taylor expansion of the sequence read data 114. Example transforms are described, for instance, in Farge, 24 Annu. Rev. Fluid Mech. 395-457 (1992), which is incorporated by reference herein its entirety.

The preprocessor 116 may perform at least one of normalizing, smoothing, or scaling to generate the processed endpoint data 118. For instance, the preprocessor 116 may normalize the endpoint data and smooth the normalized endpoint data to generate normalized and smoothed endpoint data. In some examples, the preprocessor 116 may additionally scale the normalized and smoothed endpoint data. In various instances, the preprocessor 116 may transform the endpoint data before or after performing any other preprocessing techniques described herein. In some cases, the preprocessor 116 may perform some or all of the processes described herein in any order in order to generate the processed endpoint data 118.

According to various implementations, the processed endpoint data 118 represents at least one locus-of-interest indicated by the sequence read data 114. In some examples, the preprocessor 116 determines at least one gene-of-interest based on the condition. For instance, examples of genes with potential relevance to a determination of whether the subject 102 has a type or subtype of cancer include A2M, ABCA6, ABCB1, ABCC2, ABCC9, ABI1, ABL1, ABL2, ACACA, ACLY, ACRBP, ACSL3, ACSL6, ACTA2, ACTG1, ACTG2, ACTN1, ACTR3B, ACVR1, ACVR1C, ACVRL1, ADAM12, ADAM19, ADAM2, ADCY7, ADGRB1, ADGRB3, ADGRF5, ADGRL4, ADRB2, AF10, AFF1, AFF3, AFF4, AFP, AGR2, AGR3, AHR, AIFM3, AKT1, AKT2, AKT3, ALDH2, ALK, ALOX12, AMZ1, ANGPT1, ANGPT2, ANLN, ANPEP, ANXA1, ANXA2, APC, APCDD1, APEX2, APH1A, APLN, APOBEC3A, APOBEC3B, APOBR, APOL6, APP, APPBP2, AR, AREG, ARF1, ARG2, ARHGAP15, ARHGDIA, ARID1A, ARID1B, ARID3A, ARNT, ARNT2, ASAP2, ASB13, ASCL2, ASGR2, ASTE1, ASXL1, ATAD2, ATIC, ATM, ATP2C1, ATP8A1, ATP8B2, ATR, AURKA, AURKB, AVPR1A, AXIN2, AXL, B2M, B3GNT5, BAALC, BAG1, BAG2, BAGE4, BAK1, BAMBI, BAP1, BASP1, BATF, BATF3, BAX, BAZ2B, BCAM, BCAR1, BCAR3, BCAS1, BCL10, BCL11A, BCL11B, BCL2, BCL2A1, BCL2L1, BCL2L11, BCL3, BCL6, BCL7A, BCL8, BCL9, BCOR, BCR, BIN2, BIRC3, BIRC5, BLK, BLM, BLNK, BLVRA, BMF, BMP2, BMP4, BMPR1A, BMPR1B, BNC2, BRAF, BRCA1, BRCA2, BRD3, BRD4, BRDT, BRINP3, BRIP1, BRPF1, BTG1, BTG3, BTK, BTLA, BUB1, BUB1B, C10orf35, C11orf30, C15orf48, CIRL, C3, C5, C5AR2, CA4, CAGE1, CALB2, CALML3, CALR, CAMTA1, CANX, CASP1, CASP3, CASP8, CASP9, CBFA2T3, CBFB, CBL, CBLC, CCDC140, CCDC50, CCL11, CCL13, CCL14, CCL17, CCL18, CCL19,CCL2, CCL20, CCL21,CCL3,CCL4, CCL5, CCL8, CCNA2, CCNB1, CCNB2, CCND1, CCND2, CCND3, CCNE1,CCNE2, CCNG2,CCR4, CCR5, CCR7, CCR8, CCRL2, CCSER2, CD14, CD163, CD19, CD1A, CDB, CD1D, CD1E, CD2, CD209, CD22, CD226, CD244, CD247, CD248, CD27, CD276, CD28, CD33, CD34, CD36, CD38, CD3D, CD3E, CD3G, CD4, CD40, CD40LG, CD44, CD46, CD47, CD5, CD6, CD63, CD68, CD7, CD70, CD74, CD79A, CD79B, CD80, CD81, CD84, CD86, CD8A, CD8B, CD9, CD93, CD96, CDC20, CDC25C, CDC45, CDC6, CDCA3, CDCA5, CDCA7, CDCA7L, CDCA8, CDH1, CDH3, CDH5, CDHR1, CDK2, CDK4, CDK6, CDK8, CDKN1A, CDKN1B, CDKN1C, CDKN2A, CDKN2AIP, CDKN2B, CDKN2B-AS1, CDKN2D, CDKN3, CDT1, CDX2, CEACAM1, CEACAM3, CEACAM5, CEACAM8, CEBPA, CEBPB, CELSR2, CENPA, CENPF, CENPM, CEP110, CEP55, CES1, CES2, CFD, CHAF1B, CHEK1, CHEK2, CHN1, CHUK, CIC, CIITA, CITED4, CLCA2, CLDN18, CLDN3, CLDN4, CLDN5, CLDN6, CLDN7, CLEC10A, CLEC14A, CLEC4C, CLEC5A, CLEC9A, CLIC2, CLIC4, CLTC, CMKLR1, CMPK2, CNN1, CNTNAP2, COL15A1, COL18A1, COL1A1,COL1A2, COL3A1, COL4A1, COL4A2, COL6A3, COL7A1, COPB2, CPA3, CRAT, CREB1, CREB3L1, CREB3L2, CREBBP, CRKL, CRLF2, CRNDE, CRYAB, CSF1, CSF1R, CSF2, CSF3R, CSMD1, CSNK1E, CSNK1G2, CST7, CT45A1, CT45A2, CT45A3, CT62, CTAG1A, CTAG1B, CTAG2, CTAGE1, CTGF, CTLA4, CTNNB1, CTNNBIP1, CTPS1, CTPS2, CTSV, CTSW, CUX1, CX3CL1, CXCL1, CXCL10, CXCL11, CXCL12, CXCL13, CXCL2, CXCL3, CXCL6, CXCL8, CXCL9, CXCR1, CXCR2, CXCR4, CXCR5, CXCR6, CXXC5, CYB5R2, CYBB, CYLD, CYP4F3, DCAF12, DCLK1, DCN, DDB2, DDIT3, DDIT4, DDR1, DDR2, DDX10, DDX21, DDX4, DDX58, DDX6, DEK, DENND3, DEPTOR, DHH, DHX58, DIDO1, DIRC2, DKK1, DKK2, DKK4, DLC1, DLL3, DLL4, DMBT1, DMD, DNMT1, DNMT3A, DOCK5, DOT1L, DRAM1, DSC2, DSCR8, DTL, DTX1, DTX2, DTX3L, DUSP1, DUSP18, DUSP22, DUSP6, DVL1, E2A, E2F1, E2F4, E2F5, EBF1, ECSCR, ECT2, EDNRB, EGF, EGFR, EGLN3, EGR1, EGR2, EIF4A2, ELF4, ELF5, ELK4, ELL, ELN, EMCN, EME1, EML4, EML6, ENL, ENTPD1, EOMES, EP300, EP400, EPCAM, EPHA4, EPHA7, EPOR, EPS15, ERAP1, ERAP2, ERBB2, ERBB3, ERCC1, ERCC2, ERCC3, ERCC4, ERCC5, EREG, ERG, ERN2, ESM1, ESR1, ETO, ETS1, ETV1, ETV4, ETV5, ETV6, EWSR1, EXO1, EZH2, F11R, FAM101B, FAM123B, FAM171B, FAM26F, FAM46A, FAM64A, FANCA, FANCB, FANCC, FANCD2, FAP, FASN, FAT2, FBXW11, FBXW7, FCAR, FCGR2B, FCGR3B, FCRL2, FCRL5, FEV, FGF9, FGFBP2, FGFR1, FGFR10P, FGFR2, FGFR3, FGFR4, FGR, FKBP4, FLI1, FLNA, FLT1, FLT3, FLT3LG, FLT4, FMN1, FMN2, FMOD, FN1, FNBP1, FNIP2, FOLH1, FOLR1, FOS, FOSB, FOXA1, FOXC1, FOXM1, FOX01, FOX03, FOX04, FOX06, FOXP1, FOXP3, FPR1, FPR3, FSTL3, FUCA1, FUS, FUT4, FUT8, FZD1, FZD10, FZD2, FZD5, FZD6, FZD7, GABBR2, GADD45A, GADD45B, GAGE1, GAGE2E, GAGE6, GAGE8, GALNT10, GALNT12, GAS1, GAS7, GBP5, GIMAP5, GIMAP7, GINS2, GJA4, GLI1, GLIS2, GMFG, GMNN, GMPS, GNA12, GNG11, GNLY, GOLM1, GPA33, GPC4, GPC6, GPI, GPR143, GPR146, GPR160, GRAF, GRB7, GREB1, GRM4, GSK3B, GSTA1, GSTM1, GUSB, GZMA, GZMB, GZMH, GZMK, H2AFX, HABP2, HAMP, HAP1, HAVCR2, HBEGF, HCLS1, HCST, HDAC1, HDAC10, HDAC11, HDAC2, HDAC3, HDAC4, HDAC5, HDAC6, HDAC7, HDAC8, HDAC9, HDC, HELZ2, HERPUD1, HES1, HES2, HES4, HES5, HES6, HEY 1, HEY2, HEYL, HGF, HHIP, HIF1A, HIP1, HIST1H1A, HIST1H1E, HIST1H2AG, HIST1H2AI, HIST1H2BL, HIST1H3B, HIST2H2BF, HLA-A, HLA-B, HLA-C, HLA-DMA, HLA-DMB, HLA-DOA, HLA-DOB, HLA-DQA1, HLA-DQB1, HLA-DRA, HLA-DRB1, HLA-E, HLF, HMGA1, HMGA2, HMGCS2, HMMR, HOPX, HORMAD1, HOXA11, HOXB2, HPCAL1, HRAS, HRASLS, HSD11B1, HSP90AA1, HSP90AB1, HSPA4L, HSPB1, ICAM1, ICAM2, ICOS, ID1, ID2, IDO1, IFI16, IFI27, IFI35, IFI6, IFIT1, IFIT2, IFIT3, IFITM2, IFITM3, IFNG, IFNL2, IGF1, IGF1R, IGFBP1, IGFBP3, IGFBP4, IGLL5, IHH, IKBKE, IKZF1, IKZF2, IKZF3, IL10, IL11, IL12A, IL13, IL13RA2, IL15, IL16, IL17RA, IL1A, IL1B, IL1R1, IL1RN, IL21R, IL23A, IL2RA, IL3, IL33, IL3RA, IL4R, IL6, IL6R, IL6ST, IL7, IL7R, IMPDH1, INPP1, INSR, INSRR, IPO8, IQGAP3, IRF1, IRF4, IRF7, IRF8, IRGM, IRS2, IRX4, ISG20, ISY1, ITGAM, ITGAV, ITGAX, ITGB1, ITGB2, ITGB4, ITK, ITM2A, ITPKB, JAK1, JAK2, JAK3, JAML, JAZF1, JUN, KCNE3, KCNJ15, KCNK5, KCNMA1, KDM1A, KDM3B, KDM4C, KDM5C, KDM5D, KDR, KDSR, KIAA0040, KIAA0125, KIAA0319L, KIAA1462, KIAA1804, KIF13B, KIF23, KIF2B, KIF2C, KIF5B, KIFC1, KIR2DL1, KIR2DL3, KIR3DL1, KIR3DL2, KIR3DS1, KIT, KLF2, KLF4, KLK3, KLRB1, KLRC3, KLRC4, KLRD1, KLRK1, KMT5A, KRAS, KRT14, KRT17, KRT31, KRT5, KRT6A, KRTCAP3, KYNU, LAG3, LAIR1, LAMB1, LASP1, LATS1, LATS2, LCK, LCN2, LCP1, LDHB, LEF1, LGALS2, LGALS3, LILRB5, LIMD1, LIMK2, LINC-ROR, LINC00598, LIPH, LIPI, LMNA, LMO1, LMO2, LMO3, LMO4, LOC100506207, LOC100507346, LOC100507424, LPP, LRMP, LRP1, LRP8, LRRC15, LTF, LTK, LUZP4, LY6E, LY6G6D, LYL1, LZTR1, MACC1, MAF, MAFB, MAGEA1, MAGEA10, MAGEA11, MAGEA12, MAGEA2B, MAGEA3, MAGEA4, MAGEA5, MAGEA6, MAGEA8, MAGEA9B, MAGEB1, MAGEB10, MAGEB16, MAGEB17, MAGEB18, MAGEB2, MAGEB3, MAGEB4, MAGEB5, MAGEB6, MAGEC1, MAGEC2, MAGEC3, MALAT1, MALT1, MAML2, MAML3, MAP2, MAP2K1, MAP2K3, MAP3K7, MAP3K8, MAP4K4, MAPK1, MAPK3, MAPKAPK2, MAPT, MARK1, MASP2, MAST1, MAST2, MASTL, MB21D1, MBTD1, MCAM, MCL1, MCM10, MCM2, MCM4, MCM6, MDC1, MDM2, MDS2, MECOM, MEF2C, MEF2D, MEG3, MEGF9, MELK, MEN1, MEST, MET, METRNL, MFAP4, MFAP5, MGA, MGMT, MGST2, MIA, MIAT, MICB, MIR100, MITF, MKI67, MKL1, MKL2, MLF1, MLH1, MLL, MLL2, MLL3, MLPH, MME, MMP11, MN1, MNX1, MOCOS, MPZL3, MRAS, MRE11A, MRVI1, MS4A1, MS4A2, MS4A4A, MSH2, MSH6, MSI2, MSMB, MSN, MST1R, MTAP, MTCP1, MTHFD1L, MTOR, MUC1, MUC16, MUTYH, MVP, MX1, MX2, MYB, MYBL2, MYC, MYCL1, MYCN, MYCT1, MYD88, MYH11, MYH9, MYST3, NAB2, NAT1, NAV3, NBEA, NBN, NCAM1, NCOA2, NCOR1, NCR1, NDC80, NDE1, NDRG1, NEAT1, NECTIN1, NECTIN2, NECTIN3, NEK1, NEK2, NEK6, NELL2, NF1, NF2, NFATC2, NFE2L2, NFIC, NFKB2, NID2, NIN, NKD1, NKG7, NKX3-1, NLK, NONO, NOS1, NOS1AP, NOTCH1, NOTCH2, NOTCH3, NOTCH4, NPAS2, NPM1, NR4A3, NRAP, NRARP, NRAS, NRG1, NRG2, NRP1, NRP2, NRTN, NSD1, NT5C3A, NT5E, NTRK1, NTRK2, NTRK3, NUF2, NUMA1, NUMBL, NUP214, NUP98, NUTM1, NUTM2A, NXF2B, NXPH3, OAS3, OASL, ODC1, OGN, OLFM1, OLFM4, OLIG2, ORAI2, ORC6, P2RY8, PADI2, PAFAH1B2, PAGE5, PAK2, PAK4, PALB2, PAMR1, PARP1, PARP12, PARP14, PAX3, PAX5, PAX7, PAX8, PBK, PBX1, PBX3, PCDH17, PCSK1, PDCD1, PDGFA, PDGFB, PDGFD, PDGFRA, PDGFRB, PDIA3, PDL1, PDL2, PDZK1IP1, PECAM1, PFN2, PGR, PHF1, PHF11, PHGDH, PHLPP1, PICALM, PIK3CA, PIK3CD, PIK3CG, PIK3R1, PIK3R2, PIM2, PIM3, PKN1, PLA2G7, PLAC8, PLAG1, PLAGL2, PLCB4, PLEK2, PLEKHA4, PLEKHB1, PLK2, PLPP3, PLVAP, PMEPA1, PML, PMS1, PMS2, PNOC, PNPLA7, PODXL, POLD1, POLE, POU2F2, POU5F1, PPARG, PPM1J, PPP1R13L, PRDM15, PRDM16, PRF1, PRKACA, PRKACB, PRKACG, PRKCA, PRKCB, PRMT1, PRMT5, PRND, PROM1, PRPF6, PRPF8, PSAT1, PSCA, PSD3, PSENEN, PSIP1, PSMB10, PSMB8, PSMB9, PSME1, PTCH1, PTCH2, PTCRA, PTEN, PTGDS, PTGER2, PTGER4, PTGS2, PTPN1, PTPN11, PTPN22, PTPRB, PTPRC, PTPRK, PTPRO, PTPRZ1, PTRF, PTTG1, PUM1, PVR, PVRIG, PXDC1, R3HDM1, RAB23, RAB27A, RAB29, RAC1, RAD50, RAD51, RAD51AP1, RAD51C, RAD51L1, RAD51L3, RAD52, RAD54L, RAF1, RAPGEFL1, RARA, RASGRF1, RASIP1, RASSF6, RB1, RBL1, RBM24, RBP7, RBX1, RECQL4, REG4, RELA, RERG, RET, RGCC, RGS10, RGS16, RGS2, RHOA, RHOH, RHOJ, RIT1, RNF13, ROBO4, ROCK2, ROPN1, ROPN1B, ROR1, RORA, RORC, ROS1, RP1, RPL23, RPL39L, RPS26, RPS6KA1, RPS6KB1, RPSAP52, RRAGC, RRAS, RRM2, RSAD2, RSPO2, RSPO3, RUNDC2A, RUNX1, RUNX2, RUNX3, S100A12, S100A8, SIPR2, SAA1, SAGE1, SAMD9L, SAP30, SCD, SCD5, SCML4, SCUBE2, SDC1, SDHA, SDHB, SDHC, SDHD, SEC31A, SELL, SELP, SEMA3E, SEMA4B, SEMA4C, SEMA6D, SEMA7A, SEPT12, SEPT5, SEPT6, SEPT9, SEPW1, SERPINA9, SERPINB13, SERPINB2, SERPINB5, SERPINE1, SERPINF1, SESN1, SESN2, SESN3, SET, SF3B1, SFRP1, SGK3, SH2D1A, SH2D1B, SH2D2A, SH3BP5, SH3GL1, SH3PXD2A, SHCBP1, SHISA5, SHISA8, SHOC2, SIGLEC5, SKP1, SLAMF1, SLC16A3, SLC1A2, SLC22A8, SLC39A6, SLC40A1, SLC45A3, SLC7A8, SLC9A3R1, SLCO2A1, SLFN11, SLIT2, SMAD2, SMAD3, SMAD4, SMAD9, SMARCB1, SMURF2, SNAI1, SNRNP70, SNW1, SOCS1, SOS1, SOS2, SOX11, SOX17, SOX18, SOX9, SP2, SPANXA1, SPANXB1, SPANXC, SPARC, SPARCL1, SPIB, SPINK1, SPN, SPP1, SPRY4, SRC, SRD5A1, SREBF1, SRSF3, SS18, SSPO, SSX1, SSX2, SSX2B, SSX3, SSX4, SSX5, ST3GAL2, STAT1, STAT3, STAT4, STAT6, STAU2, STEAP1, STEAP4, STIL, STK11, STON1, SULF2, SULT1A1, SUV39H2, SYCP1, SYCP3, SYK, TACSTD2, TAF15, TAGAP, TAGLN, TAL1, TAL2, TAP1, TAP2, TAPBP, TBC1D10C, TBC1D4, TBC1D9, TBL1XR1, TBX21, TCF12, TCF4, TCF7L1, TCF7L2, TCL1, TCL6, TDG, TDGF1, TDRD7, TEAD1, TEC, TEK, TENM3, TERC, TERT, TET1, TET2, TET3, TFCP2L1, TFE3, TFEB, TFF1, TFG, TFPT, TFRC, TGFB1, TGFB2, TGFB3, TGFBI, TGFBR1, TGFBR2, THADA, THBD, THBS1, THY1, TIAM1, TIE1, TIGIT, TIMP3, TLL1, TLR2, TLR3, TLX1, TLX3, TMEM173, TMEM38A, TMEM45B, TMEM55B, TMPRSS2, TNF, TNFRSF10C, TNFRSF11A, TNFRSF14, TNFRSF17, TNFRSF1A, TNFRSF1B, TNFRSF25, TNFRSF6, TNFRSF8, TNFRSF9, TNFSF10, TNFSF11, TNFSF12, TNFSF13B, TNFSF4, TNFSF9, TNKS, TNKS2, TNS1, TOP1, TOP2A, TP53, TP53BP1, TP53INP1, TP53INP2, TP63, TP73, TPM1, TPM2, TPM3, TPM4, TPSAB1, TPSB2, TPST1, TPX2, TRAT1, TREM2, TREX1, TRIM2, TRIM24, TRIM56, TRIP11, TRPS1, TSC1, TSC2, TSHR, TTC39B, TTK, TTL, TTTY14, TTYH1, TWIST1, TYK2, TYMS, UBA7, UBE2C, UBE2T, UBXN4, UGT8, UNC5B, UPK1A, UPP1, USP44, USP6, USP8, VAV3, VCAM1, VCL, VEGFA, VEGFB, VEGFC, VGLL1, VHL, VIM, VNN3, VPREB1, VWF, WASH5P, WBSCR17, WHSC1, WHSC1L1, WIF1, WNT11, WNT16, WNT2, WNT5B, WNT7A, WNT7B, WNT8B, WT1, WWTR1, XCL1, XCL2, XIST, XPA, XPO1, YAP1, YWHAE, YY1, ZAP70, ZBP1, ZBTB16, ZBTB46, ZC3H13, ZC3HAV1, ZEB1, ZEB2, ZIC2, ZMAT3, ZMYM2, ZNF384, ZNF521, ZNF608, ZNF703, ZNF750, or ZNRF3. In some cases, the genes include at least one estrogen receptor (ER) gene and/or at least one progesterone receptor (PR) gene. In some cases, the genes include one or more of ABL, ALK, ALL, ATRX, AXIN1, B4GALNT1, BAFF, BARD1, BCL2, BCL2L2, BCORL1, BRAF, BRCA, BTG2, BTK, CARD11, CD19, CD20, CD274, CD3, CD30, CD319, CD38, CD52, CDC73, CDK12, CDK4, CDK6, CDKN2C, CML, CRACC, CS1, CTCF, CTLA-4, CTNNA1, CUL3, CUL4A, CYP17A1, DAXX, dMMR, EGFR, EMSY, EP300, EPHB1, EPHB4, ERBB1, ERBB2, ERCC4, EZR, FAM46C, FANCL, FAS, FGF12, FGF14, FGF19, FGF23, FGF3, FGF4, FGF6, FGFR1-3, FH, FLT1, FLT3, FLT3, FOXL2, FUBP1, GABRA6, GATA3, GATA4, GATA6, GD2, GID4, GNA11, GNA13, GNAQ, GNAS, H3F3A, HDAC, HER1, HER2, HR, HSD3B1, IDH1, IDH2, IDH2, IL-1β, IL-6, IL-6R, INPP4B, IRF2, JAK1, JAK2, JAK3, KDM6A, KEAP1, KIT, KLHL6, KMT2A, KMT2D, KRAS, LYN, MAP2K2, MAP2K4, MAP3K1, MAP3K13, MDM4, MED12, MEF2B, MEK, MERTK, MET, MKNK1, MPL, MSH3, MSI-H, mTOR, MYCL, NFKBIA, NKX2-1, NT5C2, PARK2, PARP, PARP2, PARP3, PBRM1, PD-1, PD-L1, PDCD1LG2, PDGFR, PDGFRα, PDGFRβ, PDK1, PI3K8, PIGF, PIK3C2B, PIK3C2G, PIK3CB, PIM1, PPP2R1A, PPP2R2A, PRDM1, PRKAR1A, PRKCI, PTCH, QKI, RAD21, RAD51B, RAD51D, RAF, RANKL, RBM10, REL, RET, RICTOR, RNF43, ROS1, RPTOR, SDC4, SETD2, SGK1, SLAMF7, SLC34A2, SMARCA4, SMO, SNCAIP, SOX2, SPEN, SPOP, STAG2, SUFU, TBX3, TIPARP, TNFAIP3, TYRO3, U2AF1, VEGF, VEGFA, VEGFB, XRCC2, or ZNF217. In some examples, the genes include one or more of TP53, CTNNNB1, L1CAM, PTEN, POLE, MKI67, FAT3, TAF1, ZFHX3, RPL22, SPTA1, FAM135B, CSMD3, GIGYF2, CSDE1, MLL4, ATR, CTNNB1, USH2A, LIMCH1, RRN3P2, FBXW7, CDH19, USP9X, COL11A1, BCOR, ARID1A, ZNF770, ARID5B, SLC9A11, KRAS, PNN, INPP4A, CTCF, CHD4, AMY2B, RBMX, PPP2R1A, TNFAIP6, PIK3R1, SGK1, HOXA7, METTL14, HPD, MIR1277, CCND1, MECOM, NFE2L2, or ESR1.

In various instances, the preprocessor 116 may identify the at least one locus-of-interest using benchmark sequence read data 124. Utilizing the benchmark sequence read data 124, in various cases, can enable identification of genomic regions associated with the condition. In various implementations, the endpoint data may be limited to the at least one locus-of-interest, or the at least one locus-of-interest may be assigned a greater weight than other genomic regions, in order to improve identification of features in the sample 108 associated with the condition of the subject 102.

The benchmark sequence read data 124 is, in some examples, indicative of nucleic acid molecules in benchmark samples collected from benchmark subjects 126. The benchmark subjects 126, in various cases, have the condition. For example, the benchmark subjects 126 may have a cancer type or a cancer subtype. In some examples, the benchmark subjects 126 have high-shedding tumors associated with the cancer type or the cancer subtype. For instance, the benchmark samples may be associated with a non-zero ctDNA tumor fraction estimate. In various cases, the benchmark subjects 126 have a tumor classification. In various cases, the benchmark subjects 126 have a non-cancer condition. The benchmark subjects 126, in some instances, omit the subject 102.

The benchmark sequence read data 124 is indicative of benchmark endpoint data of the nucleic acid molecules in the benchmark samples. The benchmark endpoint data (e.g., benchmark endpoint counts), in various cases, are with respect to reference sequence(s) (e.g., a reference genome). In some implementations, the endpoint data and the benchmark endpoint data are determined with respect to the same reference sequence(s). In some examples, the benchmark endpoint data may be normalized and/or smoothed. In some examples, the preprocessor 116 identifies or generates benchmark endpoint data based on the benchmark sequence read data 124. In various cases, the preprocessor 116 determines benchmark distance metrics that are indicative of the difference between the baseline endpoint data and the benchmark endpoint data. Accordingly, the benchmark distance metrics may be indicative of differences between sequence read data of subjects with the condition and sequence read data of subjects without the condition. The benchmark distance metric may be indicative of a likelihood that a genomic position is associated with the condition.

In various cases, the benchmark distance metrics are based on the mean and/or the standard deviation of the benchmark endpoint data. In some instances, the benchmark distance metrics are indicative of a difference between a mean of the benchmark endpoint data and a mean of the baseline endpoint data at a genomic position. In various implementations, the benchmark distance metrics may include a z-score. For instance, the preprocessor 116 may determine a difference between the mean of the benchmark endpoint data and the mean of the baseline endpoint data at a genomic position. The difference may be divided by a standard deviation of the baseline endpoint data at the genomic position to determine the z-score.

The preprocessor 116, in some examples, identifies the at least one locus-of-interest by analyzing the benchmark distance metrics. For instance, the preprocessor 116 may compare the benchmark distance metrics to a threshold. For instance, in the case that the benchmark distance metrics include absolute values of z-scores, the threshold may be in a range of about 1.5 to about 6. In particular examples, the threshold may be in a range of about 4 to about 5. The preprocessor 116 may identify one or more genomic positions associated with benchmark distance metrics that are greater the threshold. In some examples, the preprocessor 116 may identify the at least one locus-of-interest based on a number or a relative number (e.g., a fraction) of the genomic positions in the locus that are associated with benchmark distance metrics that are greater than the threshold.

In some examples, the preprocessor 116 may analyze the genomic positions associated with benchmark distance metrics that are lower than the threshold. For instance, the benchmark distance metrics may be inversely correlated to the difference between the sequence read data of subjects with the condition and subjects without the condition. In some examples, the benchmark distance metrics include positive and negative z-scores. The threshold, for instance, may be in a range of −6 to −1.5 and/or a range of 1.5 to 6. The preprocessor 116 may identify the at least one locus-of-interest based on a number or a fraction of the genomic positions in the locus that are associated with benchmark distance metrics that are less than the threshold. In various implementations, the preprocessor 116 may use one or more statistical tests to determine and/or analyze the benchmark distance metrics in order to identify the at least one locus-of-interest.

In particular implementations, the preprocessor 116 generates first and second benchmark distance metrics associated with the condition. For example, the preprocessor 116 may generate first benchmark distance metrics based on first benchmark subjects 126 with a first subtype of a cancer. The preprocessor 116, in some examples, generates second benchmark distance metrics based on second benchmark subjects 126 with a second subtype of the cancer. The baseline sequence read data 120 is, in various instances, based on baseline subjects 122 without the cancer and/or baseline subjects 122 with low-shedding tumors associated with the cancer. For example, the baseline samples may be derived from breast cancer patients with low-shedding tumors and/or subjects who do not have breast cancer. In various instances, the first benchmark subjects 126 include hormone receptor-positive (HR+) breast cancer patients. In various instances, the second benchmark subjects 126 include triple negative breast cancer patients. In some cases, the baseline sequence read data 120 is based on baseline subjects 122 who do not have the first subtype or the second subtype of the cancer. The baseline subjects 122 may have a third subtype of the cancer. In some cases, the baseline sequence read data 120 is based on baseline subjects 122 with low-shedding tumors associated with the first subtype or the second subtype of the cancer.

The preprocessor 116 may compare the first and second benchmark distance metrics to identify, for instance, at least one locus-of-interest for the first subtype of the cancer and/or for the second subtype of the cancer. For instance, the preprocessor 116 may perform a Mann-Whitney U test, a t-test or another statistical test to compare the first and second benchmark distance metrics. The preprocessor 116, in some examples, compares the results of the statistical test to a threshold to identify the at least one locus-of-interest. In various cases, the processed endpoint data 118 is indicative of the at least one-locus of-interest. For instance, the processed endpoint data 118 may include the at least one locus-of-interest. In some examples, the processed endpoint data 118 is limited to the at least one locus-of-interest. In some examples, the preprocessor 116 may assign a greater weight to at least one locus-of-interest in the processed endpoint data 118. Accordingly, identifying the at least one locus-of-interest may reduce the computing resources involved in analyzing the sequence read data 114 to identify the condition of the subject 102.

According to various implementations of the present disclosure, the processed endpoint data 118 may be indicative of one or more of the endpoint positions of the nucleic acid molecules 110, left endpoint positions of the nucleic acid molecules 110, or right endpoint positions of the nucleic acid molecules 110, or a length of the nucleic acid molecules 110. For instance, the processed endpoint data 118 may indicate a length of fragments (e.g., a mean length, a median length, or the like) with an endpoint, a left endpoint, a right endpoint, or a midpoint at each genomic position. In various cases, the preprocessor 116 determines, based on the left and right endpoint positions, the fragment length of the nucleic acid molecules 110. In various cases, the preprocessor 116 may convert the processed endpoint data 118 into a frequency distribution (e.g., a two-dimensional visual representation of the preprocessed endpoint counts with respect to genomic position).

A feature selector 128 identifies input features 130 of the nucleic acid molecules 110 by analyzing the sequence read data 114 and/or the processed endpoint data 118. In various examples, the input features 130 include the processed endpoint data 118. The processed endpoint data 118 may be normalized, smoothed, scaled, or a combination thereof. In various cases, the input features 130 include an indication of the at least one locus-of-interest, the baseline sequence read data 120, the benchmark sequence read data 124, or a combination thereof. In some cases, the feature selector 128 identifies, calculates, or otherwise determines the input features 130 based on the sequences of the nucleic acid molecules 110 indicated in the sequence read data 114. One or more types of features are identified by the feature selector 128. In various implementations, the input features 130 are genomic features. That is, the input features 130 may be derived from the sequence read data 114 in addition to the processed endpoint data 118. In some examples, the input features 130 may be derived from transformed data corresponding to the sequence read data 114 and/or the processed endpoint data 118. In various cases, the input features 130 include a ctDNA tumor fraction estimate of the sample 108.

In some examples, the feature selector 128 includes one or more machine learning (ML) models configured to identify features of the sequence read data 114 and/or the processed endpoint data 118 associated with the condition. For instance, the feature selector 128 may perform image processing techniques in order to generate the input features 130. In some cases, the feature selector 128 generates a digital image based on the processed endpoint data 118. For example, the feature selector 128 may generate a spectrogram or other graphical representation of the transformed data. In some cases, the feature selector 128 generates the input features 130 by analyzing the image of the transformed data. The feature selector 128 may include a convolutional neural network (CNN) that generates the input features 130 in response to receiving the image representative of the processed endpoint data 118. For instance, the pixel intensities may be indicative of a distribution of the DNA fragments indicated by the processed endpoint data 118.

According to various examples, the CNN may include multiple blocks and/or layers that are each defined by a kernel (e.g., a digital image filter). Each block and/or layer may be configured to convolve and/or cross-correlate the kernel with pixels of an input image, thereby generating an output image. In some cases, the blocks and/or layers are arranged in series, such that the input image of one block and/or layer may be the output image of another block and/or layer. Each block and/or layer may further be defined according to a receptive field of its kernel and/or a stride size of the kernel.

In some examples, the CNN of the feature selector 128 is pretrained. For example, the values of the kernel of each block and/or layer may be optimized based on training data prior to receiving the image of the processed endpoint data 118. In some examples, the training data includes other images of other transformed data, as well as manually obtained indications of the types of input features that the CNN is being trained to identify. The CNN, for instance, may be trained using a supervised learning technique. Because the CNN is pretrained, the CNN may be configured to output the input features 130 in response to receiving the image of the processed endpoint data 118.

In various cases, the input features 130 are derived based on fragments in the nucleic acid molecules 110, and are therefore referred to as “fragmentomic features.” Examples of fragmentomic features include endpoint positions of the fragments in a reference genome (e.g., right endpoints, left endpoints, etc.), endpoint counts at positions within the reference genome (e.g., right endpoint counts, left endpoint counts, etc.), fragment lengths, end motifs, relative read depths of the fragments, the presence of one or more variants in the fragments, or any combination thereof. Fragmentomic features can be expressed in the spatial domain, in an alternate domain, in a preprocessed form, or any combination thereof.

To categorize the condition of the subject, a predictive model 132 is configured to determine one or more condition indicators 134 based on the input features 130. The predictive model 132, for example, may include one or more mathematical and/or computer-based models that are configured to predict the condition indicator(s) 134 based on the input features 130. For instance, the predictive model 132 may include a regression model, threshold rule, confidence interval, or other type of statistical model capable of categorizing the lesion 104 or a non-cancer condition of the subject 102 based on the input features 130. In various cases, the predictive model 132 includes at least one classifier configured to generate the condition indicator(s) 134 based on the input features 130.

In various implementations, the predictive model 132 includes at least one trained ML model configured to output the condition indicator(s) 134 in response to receiving the input features 130 in input data. For example, parameters of the ML model(s) may have been previously optimized based on training data including features of individuals within a population omitting the subject 102. For instance, the ML model(s) was trained using an unsupervised or semi-supervised learning technique, wherein the parameters were optimized to categorize (e.g., cluster) the features of the population. In some cases, the ML model(s) was trained using a supervised learning technique, wherein the training data further included ground truth conditions (e.g., a tumor classification) of the individuals in the population, such that the parameters were optimized to minimize a loss between predicted conditions generated by the ML model(s) based on the features of the population and the ground truth conditions of the cancers experienced by the individuals in the population. To increase training robustness, the population represented by the training data may include individuals without the condition (e.g., a cancer subtype, a tumor classification), individuals with a low-shedding tumor associated with the condition (e.g., a cancer type), individuals without cancer, as well as individuals with a variety of types of presentations of the condition. According to some implementations, the predictive model 132 may be configured to identify or further analyze the at least one locus-of-interest associated with the condition of the subject 102. For instance, the predictive model 132 may be configured to analyze the processed endpoint data 118 by selecting a subset of the genomic loci associated with the condition based on the training data.

Various types of ML models can be included in the predictive model 132, such as a neural network (e.g., a CNN), a nearest-neighbor model, a regression analysis model, a clustering model, a principal component analysis model, a gradient boosting model, a random forest, a linear discriminant analysis (LDA) model, or any combination thereof. In some cases, the predictive model 132 includes a hybrid model, that includes multiple types of ML models. For instance, the predictive model 132 may include a neural network and a clustering model.

In particular examples, the predictive model 132 includes a clustering model. In various implementations, the clustering model is pre-trained based on training data that includes population features. According to various implementations, the population features include genomic features and/or additional biomarker data of the population. In some cases, the population features further include one or more known conditions and/or prognostic classifications of the population. In various implementations, at least one computing device is configured to cluster the population features. The clustering model, for instance, stores, includes, or otherwise indicates the determined clusters.

In various examples, the population characteristics are defined in a multi-dimensional feature space. In various cases, the feature space has n dimensions (e.g., a dimensionality value of n), wherein n corresponds to the number of feature types included in the population features. For example, one dimension may correspond to a number of genomic positions in the processed endpoint data 118 that exceed a threshold, another dimension may refer to a distance metric representing a similarity between the processed endpoint data 118 and pre-classified endpoint data based on a sample obtained from an individual with a particular type of cancer, and so on. In various cases, data objects representing the population features of the population are plotted or otherwise defined in the feature space. In some examples in which n is greater than two, the data objects are projected onto an m-dimensional feature space using multi-dimensional scaling, wherein m is between 1 and n−1 (inclusive). Multi-dimensional scaling can be achieved using various techniques. For instance, multi-dimensional scaling can be performed using at least one of a statistical method (e.g., t-distributed stochastic neighbor embedding (t-SNE), uniform manifold approximation and projection (UMAP), representation learning (e.g., principal component analysis (PCA), independent component analysis (ICA), etc.), ML-based latent space learning (e.g., autoencoders, transformers, generative adversarial networks, etc.). Accordingly, in some cases, the data objects can be visualized in a Cartesian coordinate system.

Within the feature space (whether it has two or more than two dimensions), the data objects are separated from each other by distances. Various types of distances can be utilized in implementations of the present disclosure. For example, the distances may include Euclidian distances, Manhattan distances, Hamming distances, Minkowski distances, Chebyshev distances, or any combination thereof.

Various clustering techniques can be utilized to generate the clustering model. For instance, the clusters may be generated using k-means clustering, density-based clustering, centroid-based clustering, spectral clustering, distribution-based clustering, hierarchical clustering, or any combination thereof. In some implementations, the clustering model is generated by performing hierarchal clustering on the data objects representing the population features. In various cases, the clusters include two or more data objects that are within proximity of each other (e.g., within a predetermined distance of one another) in the feature space. For instance, a cluster may include two or more data objects that are within a predetermined distance (e.g., Euclidian distance) of one another in the feature space. In some implementations, a data object is included in a cluster if the data object is within an appropriate distance of a linkage criterion representing one or more data objects that are already defined within the cluster. Various implementations of the present disclosure utilize one or more linkage criteria, such as a single-linkage criterion, a complete-linkage criterion, an average-linkage criterion (e.g., a weighted average criterion, an unweighted average criterion), a centroid-linkage criterion, a median linkage criterion, a Ward linkage criterion, a minimum error sum of squares criterion, a min-max criterion, a Hausdorff linkage criterion, a medoid linkage criterion, a minimum energy clustering criterion, or any combination thereof.

In some cases, agglomerative clustering is used to generate the clusters. For example, initially, each data object is defined within the feature space without clustering. Subsequently, pairs of adjacent data objects may be clustered together. In some examples, the process of generating a cluster based on independent data objects in a feature space, or of adding a data object to an existing cluster, may be referred to as “merging.”

In some examples, divisive clustering is used to generate the clusters. For example, the data objects may be defined into a single cluster in the feature space. Subsequently, the single cluster may be divided into multiple clusters. In some instances, the process of dividing a preliminary cluster into multiple subsequent clusters, or of removing a data object from a cluster, may be referred to as “splitting.”

In various cases, each cluster is defined according to a boundary (also referred to as a “border”). In some implementations, data objects outside of the boundary of a cluster are not part of the cluster. Data objects inside of the boundary of the cluster are part of the cluster. Depending on the data objects, the linkage criterion, the feature space, and other characteristics of the training data, the clusters may have irregular shapes within the feature space. In various cases, the clustering model includes the boundaries of the clusters generated based on the data objects defined by the population features.

According to various cases, each cluster in the clustering model is associated with one or more characteristics. The characteristic(s), for instance, are associated with the presence or absence of the condition in the samples associated with the cluster. In some cases, at least one characteristic is defined in at least one dimension of the feature space, such that the clusters are defined according to the condition (e.g., cancer type, tumor classification, etc.). In some examples, the population features used to define the clusters include characteristics that are beyond the mere categorization of the presence or absence of the condition in the population. Once the clusters are generated based on non-condition features (e.g., genomic features, such as fragmentomic features, and/or additional biomarker data), characteristics associated with the clusters are subsequently determined. For example, an example cluster may be defined based on the data objects representing the non-condition population features of m members of the population, wherein m is an integer that is greater than one. In various cases, characteristics of the m members of the population are determined. Common characteristics of the population (e.g., the presence or absence of the tumor classification) are determined. For example, if greater than a threshold number of the m members have the condition that is resistant to a predetermined therapy, then resistance to the predetermined therapy may be associated with the example cluster. In various cases, each cluster may be labeled with, or otherwise associated with, one or more characteristics, such as one or more pathological and/or nonpathological conditions. The one or more conditions and/or prognostic features associated with a given cluster form the characteristic(s) associated with the cluster. In various cases, each cluster in the clustering model is associated with a particular condition state (e.g., a particular cancer subtype, a particular tumor classification, etc.).

In various implementations, the condition of the subject 102 is categorized by comparing the input features 130 of the subject 102 to the clusters in the clustering model. The condition indicator(s) 134 are determined based on a comparison between the input features 130 and the clusters in the clustering model. In various cases, a data object defined by the input features 130 of the subject 102 is defined in the feature space of the clustering model. The clustering model, for instance, may determine that the data object is present within the boundary of a particular cluster that was previously defined based on the training data. In some cases, the clustering model determines that the data object is associated with a particular cluster based on a distance between the data object and the particular cluster in the feature space. In some cases, the distance is at least one of a Euclidian distance, a Manhattan distance, a Hamming distance, a Minkowski distance, a Chebyshev distance, or any combination thereof. For instance, the clustering model determines that the distance between the data object and the boundary and/or a centroid of the particular cluster is below a threshold distance. In some examples, the clustering model classifies the condition of the subject 102 into a classification associated with the particular cluster by determining that a distance between at least one data object corresponding to the population features in the cluster is below a threshold distance.

In various cases, the condition indicator(s) 134 of the sample 108 are generated using the input features 130 and the clustering model. For example, the clustering model may determine that the subject 102 is associated with one or more conditions and/or prognostic features associated with the cluster in which the input features 130 belong.

In various examples, the prognostic features may include the predicted presence or absence of the condition (e.g., a cancer type or a cancer subtype). For instance, the predictive model 132 may include a neural network configured to determine a binary output indicative of a likelihood that the subject 102 has a particular condition. In some implementations, the prognostic features include a predicted likelihood of two or more conditions. For instance, the predictive model 132 may include a multi-class classifier configured to determine a first likelihood that the subject 102 has a first condition (e.g., non-small cell lung cancer), a second likelihood that the subject 102 has a second condition (e.g., breast cancer), a third likelihood that the subject 102 has a third condition (e.g., colorectal cancer), and a fourth likelihood that the subject 102 has a fourth condition (e.g., prostate cancer). The first likelihood, the second likelihood, and the third likelihood may be normalized (e.g., to 1), such that the likelihoods are relative to each other. In various examples, the predictive model 132 includes two or more binary classifiers.

In some cases, the prognostic features include a predicted tumor classification of the subject 102. The predicted tumor classification may indicate one or more of a tissue of origin of a cancer of the subject 102, a histological tissue type of the lesion 104, a primary site designation of the lesion 104, or a genomic subtype of the cancer of the subject 102. For instance, the lesion 104 may be associated with a mixed histological tissue type, such as an adenosquamous carcinoma, a carcinosarcoma, a teratocarcinoma, a mixed mesodermal tumor, or a mixed neuroendocrine-non-neuroendocrine neoplasm. The primary site designation, in some cases, indicates whether the lesion 104 is a primary tumor or a secondary tumor. In some examples, the predicted tumor classification may indicate a tumor dependency of the subject 102.

In various cases, the prognostic features include a predicted metastasis profile of the lesion 104, a predicted resistance of the lesion 104 to a therapy, or a predicted survivability of the subject 102. The predicted metastasis profile may indicate a time (e.g., a date, a time range) when the lesion 104 will metastasize. In some examples, the condition indicator(s) 134 include a likelihood that the lesion 104 will metastasize (e.g., to the lymph nodes, or to a particular organ) by a given time or an indication that there is greater than a threshold (e.g., 80%) likelihood that the lesion 104 will metastasize by the given time.

In various examples, the prognostic features include a predicted condition (e.g., disease) of the subject 102; a predicted disease subtype of the subject 102; a predicted survivability of the subject 102; one or more predicted symptoms of the subject 102; a predicted (e.g., suggested) effective therapy to treat the predicted disease of the subject 102; a dosage of one or more therapeutic agents (e.g., biologics, chemotherapeutic agents, etc.) predicted to treat the condition of the subject 102, a predicted stage of the predicted disease of the subject 102; a predicted grade of the predicted disease of the subject 102; a predicted activity level of the subject 102 (e.g., a predicted Eastern Cooperative Oncology Group (ECOG) performance status of the subject 102); a predicted smoking history of the subject 102; a predicted breast density of the subject 102; a clinical trial that the subject 102 is predicted to qualify (e.g., be eligible) for; or a characteristic of the predicted disease of the subject. For instance, the condition indicator(s) 134 may indicate that the subject 102 is likely to qualify for a clinical trial based on an age, a gender, a disease stage, and previous treatments of the subject 102.

In some examples, the input features 130 include the fragment lengths associated with the processed endpoint data 118. For instance, the preprocessor 116 may provide, to the predictive model 132, an indication of the fragment lengths associated with the processed endpoint data 118. In some instances, the input features 130 include an indication of left and/or right endpoints associated with the processed endpoint data 118. In various implementations, the input features 130 may include a clonal hematopoiesis (CH)-status of the sample 108 and/or one or more mutations indicated by the sequence read data 114.

In various implementations, the condition indicator(s) 134 include the condition(s) and/or prognostic feature(s) determined, by the predictive model 132, to be associated with the sample 108 of the subject 102. In some examples, the condition indicator(s) 134 include a likelihood that the subject 102 has a given condition (e.g., a cancer type, a tumor classification, a non-cancer condition, etc.) or an indication (e.g., a Boolean value) that there is greater than a threshold (e.g., 90%) likelihood that the subject 102 has the given condition. In some examples, the condition indicator(s) 134 include a likelihood that the subject 102 does not have the given condition or an indication that there is greater than a threshold (e.g., 90%) likelihood that the subject 102 does not have the given condition.

In some implementations, the predictive model 132 is unable to conclusively categorize the condition of the subject 102. For example, the predictive model 132 may determine that the input features 130 of the subject 102 do not fit within any of the previously defined clusters in the clustering model. In various cases, the predictive model 132 may output an indication that that the categorization of the tumor heterogeneity is inconclusive.

A report generator 136 is configured to generate a report 138 based, at least in part, on the condition indicator(s) 134. The report 138, for example, includes consumable data that can inform the care provider(s) 106 about the conditions indicator(s) 134 of the subject 102. In various implementations, the report 138 may indicate the results of additional analyses, such as the results of a histological study, whole transcriptome sequencing, RNA sequencing, whole exome sequencing (WES), whole genome sequencing, a gene expression profiling test, a cancer (e.g., DNA) hotspot panel test, a DNA methylation test, a tumor mutational burden (TMB) test, a DNA fragmentation test, an RNA fragmentation test, a microsatellite instability (MSI) test, a tumor mutational burden (TMB) test, or a viral status test. The performance of such tests is within the ordinary skill of the art, with additional detail provided elsewhere herein. The report 138, for example, may include a genomic profile of the subject 102 based on various combinations of the above analyses and tests. The genomic profile, in various cases, includes results from a comprehensive genomic profiling test, a whole genome sequencing (WGS) test, a whole exome sequencing (WES) test, a gene expression profiling test, a cancer hotspot panel test, a DNA methylation test, a DNA fragmentation test, or an RNA fragmentation test. In some examples, the report 138 may include results of analyses performed on previously-obtained samples from the subject 102. For instance, the report 138 may indicate previous condition indicator(s) determined by the predictive model 132 based on previous sequence read data of a sample obtained from the subject 102. The report 138 may indicate a change in the condition of the subject 102. For example, the report 138 may indicate that a cancer of the subject 102 has converted from HR+ to HR-negative. In some cases, the report 138 may indicate that the HR status conversion is associated with resistance to a therapy being administered to the subject 102.

In some implementations, the report 138 indicates that a follow-up test of the subject 102 is indicated. For instance, in response to determining that the categorization of the disease is inconclusive, the report generator 136 may generate the report 138 to indicate that one or more additional tests (e.g., a histological study, genome sequencing, exome sequencing, additional DNA sequencing, RNA sequencing, transcriptome sequencing, etc.) should be performed in order to identify the condition of the subject 102. In some examples, the one or more addition tests may include diagnostic imaging, such as magnetic resonance imaging, computed tomography scan, ultrasound, X-ray, mammogram, positron emission tomography, bone scintigraphy, myelography, virtual colonoscopy, echocardiography, radiography, nuclear medicine, fluoroscopy, or single-photon emission computed tomography.

In various cases, the report 138 is output to a clinical device 140. For example, the report generator 136 transmits the report 138 to the clinical device 140. In various implementations, the clinical device 140 is a computing device that is operated by, owned by, or otherwise associated with the care provider(s) 106. For instance, the clinical device 140 may be a desktop computer, a laptop computer, a smart phone, or some other computing device associated with the care provider(s) 106. The clinical device 140, in various cases, outputs the report 138 to the care provider(s) 106. In some cases, the clinical device 140 includes a display (e.g., a screen) that visually presents the report 138. In various cases, the clinical device 140 includes a speaker that outputs a sound indicative of the report 138. The clinical device 140, in various cases, may output the information in the report 138 using one or more output mechanisms or devices.

The care provider(s) 106 may review the report 138 by interacting with the clinical device 140. The report 138, in various cases, may enhance the clinical decision-making of the care provider(s) 106. For instance, the care provider(s) 106 may prepare and/or administer a treatment to the subject 102 based on the report 138, such as drug therapy, radiation therapy, targeted therapy, vaccine therapy, stem cell transplantation, blood transfusion, physical therapy, psychiatric therapy, or surgery. For instance, the care provider(s) 106 may determine a dosage of the treatment based on the report 138. According to various implementations, the care provider(s) 106 may initiate the treatment and/or refer the subject 102 to another care provider to receive the treatment. In various cases, the care provider(s) 106 may prescribe, suggest, or administer an anticancer agent for the subject 102. For example, the care provider(s) 106 may rely on the condition indicator(s) 134 reflected in the report 138 to select a treatment that the lesion 104 is predicted to be susceptible to.

In various implementations, the care provider(s) 106 may develop a diagnosis and/or prognosis of the subject 102 based on the report 138. In various implementations, the care provider(s) 106 may communicate information in the report 138 to the subject 102.

FIG. 1 illustrates various elements that can be embodied in one or more computing devices. For example, at least a portion of the functions of the sequencer 112, the preprocessor 116, the feature selector 128, the predictive model 132, the report generator 136, or the clinical device 140 are performed by one or more processors in at least one computing device. Examples of computing devices include server computers, desktop computers, laptop computers, tablet computers, mobile phones, wearable devices, Internet of Things (IoT) devices, and the like. In various cases, instructions for performing at least a portion of the functions of these elements are stored in memory and/or in a non-transitory computer readable medium. The instructions, for instance, are executed by the processor(s).

FIG. 1 also illustrates various types of data. For example, one or more of the sequence read data 114, the processed endpoint data 118, the baseline sequence read data 120, the benchmark sequence read data 124, the condition indicator(s) 134, or the report 138, or any combination thereof, includes data. The various types of data illustrated in FIG. 1 may be stored, such as in memory or in non-transitory computer readable media. In various implementations, at least a portion of the data is transmitted or otherwise output by one or more computing devices. For example, a computing device may transmit one or more communication signals to another computing device, wherein the communication signal(s) encode at least a portion of the data. Examples of communication signals include electromagnetic signals, optical signals, ultrasonic signals, optical signals, and electrical signals. For example, communication signals can be transmitted wirelessly and/or in a wired fashion. The communication signals, for instance, are transmitted over one or more wireless channels and/or one or more wired channels (e.g., optical cabling, electrical cabling, etc.). In various cases, the communication signal(s) are transmitted over one or more communication networks. A communication network, for instance, may be defined according to one or more physical channels, such as one or more frequency spectra. In some cases, a communication network is defined according to one or more communication protocols and/or standards. Examples of communication networks include fiber optic networks, Institute of Electrical and Electronics Engineers (IEEE) networks (e.g., WI-FI™ networks, WiMAX networks, BLUETOOTH™ networks, etc.), cellular networks (e.g., a 3rd Generation Partnership Project (3GPP) radio network, such as a Long Term Evolution (LTE) network, a New Radio (NR) network; or a cellular core network such as a 3rd Generation (3G) core, a 4th Generation (4G) core, a 5th Generation (5G) core, etc.), ultrasonic networks, and the like. In some cases, the data is broadcasted from one device to multiple other devices. In some cases, the data is unicasted from one device to another device. For instance, various forms of data described herein may be transmitted via a peer-to-peer (P2P) connection.

A particular example will now be described with reference to FIG. 1. In this example, the subject 102 presents to a clinical environment due to unexplained weight loss and pain. The care provider 106 may, without ordering imaging of the subject 102, obtain the sample 108 from the blood of the subject 102. The sequencer 112 may generate sequence read data 114 based on DNA fragments within the blood sample 108 of the subject 102. For example, the sequence read data 114 may represent endpoint positions of the DNA fragments within one or more genes associated with breast cancer.

A preprocessor 116 may generate the endpoint data based on the sequence read data 114. For instance, the preprocessor 116 may generate the endpoint data by determining a number of DNA fragments associated with an endpoint position at each genomic position. The preprocessor 116 may normalize the endpoint data. The preprocessor 116, in various cases, may smooth the endpoint data using a window of 31 genomic positions centered at each genomic position of the endpoint data. In some examples, the preprocessor 116 scales the endpoint data based on baseline endpoint data indicated by baseline sequence read data 120. The baseline sequence read data 120, in various cases, is associated with baseline subjects 122 who do not have breast cancer and/or baseline subjects 122 who have low-shedding tumors associated with breast cancer (e.g., subjects whose samples have an absence of ctDNA). The preprocessor 116, in some examples, determines at least one genomic locus related to breast cancer by comparing the baseline sequence read data 120 to benchmark sequence read data 124 associated with benchmark subjects 126. The benchmark subjects 126, in some cases, have breast cancer or a particular subtype of breast cancer (e.g., HR+breast cancer, human epidermal growth factor receptor 2-positive (HER2+) breast cancer, triple negative (TN) breast cancer, or the like). The processed endpoint data 118 may be indicative of the at least one genomic locus related to breast cancer. In some examples, the preprocessor 116 compares first benchmark sequence read data 124 associated with benchmark subjects 126 who have HR+breast cancer to second benchmark sequence read data 124 associated with benchmark subjects 126 who have TN breast cancer. The preprocessor 116 may generate distance metrics in a z-score space indicative of the difference between the endpoint positions indicated by the first benchmark sequence read data 124 and the endpoint positions indicated by the second benchmark sequence read data 124. Based on the z-scores, the preprocessor 116 may identify at least one genomic locus related to HR+breast cancer and/or at least one genomic locus related to TN breast cancer. The processed endpoint data 118, in various cases, is indicative of the at least one genomic locus related to HR+breast cancer and/or the at least one genomic locus related to TN breast cancer.

The preprocessor 116, in various examples, provides the processed endpoint data 118 to the predictive model 132. The predictive model 132 is configured to determine, based on the processed endpoint data 118, whether the patient has breast cancer. In some examples, the predictive model 132 is configured to determine, based on the processed endpoint data 118, whether the patient has HR+ breast cancer or TN breast cancer. For instance, the predictive model 132 includes at least one ML model trained to identify a likelihood that the subject 102 has HR+ breast cancer based on the processed endpoint data 118. Upon analyzing the processed endpoint data 118, the predictive model 132 outputs the condition indicator 134 that indicates the subject 102 has a 98% likelihood of having HR+ breast cancer. That indication is summarized on the report 138 and output to the clinical device 140. In some cases, the condition indicator(s) 134 further include a histological tissue type of the lesion 104 and/or a genomic subtype of the cancer of the subject 102. In some examples, the condition indicator(s) 134 indicate one or more genes and/or proteins associated with survival and/or growth of the lesion 104 (e.g., a tumor dependency). For instance, the condition indicator(s) 134 may indicate that the lesion 104 is PIK3CA-dependent. Thus, the care provider 106 may inform the subject 102 of the likely diagnosis and begin discussions of treatments without performing invasive testing on the subject 102.

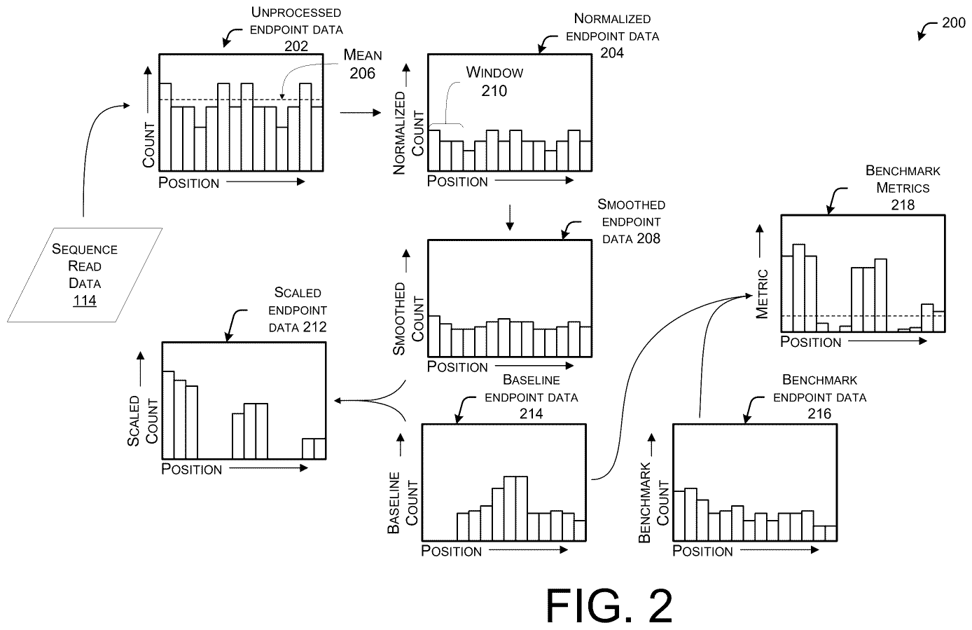

FIG. 2 illustrates example preprocessing 200 of fragmentomic data (e.g., endpoint data) for use in health-related condition classification. Different biological states, including tumor types, cell types, blood types, biomarkers, and the like, produce different patterns of fragmentation in biological patterns. However, raw endpoint density and other types of fragmentomic data can be impacted not only by the nucleic acid fragments in the sample being processed, but also by sources of artifact. These sources, for instance, include discrepancies due to low tumor fraction in the sample, sequencing errors, sequencing frequency due to bait molecule genomic location, and shearing of fragments during sample acquisition and processing. Due to the presence of these artifacts, it may be difficult to infer biologically relevant fragmentomic patterns in raw fragmentomic data.

Various implementations of the present disclosure address these and other challenges by preprocessing fragmentomic data before analysis. Example techniques described herein can remove artifact from fragmentomic data. According to various cases, preprocessing techniques described herein can enhance the accuracy, sensitivity, and specificity of various classifications performed using fragmentomic data. For instance, techniques described herein can enhance the accuracy of identifying a condition of a subject based on fragmentomic data generated based on one or more samples obtained from the subject. Techniques described herein are particularly relevant for screening techniques, wherein a sample with a relatively small amount of relevant fragments can be used to accurately assess whether the subject has the condition.

The preprocessing 200 is performed, in some examples, by the preprocessor 116 described above with reference to FIG. 1. The preprocessing 200, in various cases, includes the sequence read data 114, the baseline sequence read data 120, the benchmark sequence read data 124, and the processed endpoint data 118 described above with reference to FIG. 1. The endpoint data is illustrated as a visual two-dimensional representation of endpoint counts in FIG. 2. However, in various implementations of the present disclosure, the endpoint data may be one-dimensional or represented in another form.

The sequence read data 114 represents sequences of nucleic acid molecules in a sample obtained from a subject. One of the dimensions of the sequence read data 114, for instance, represents genomic position with respect to a reference genome. In some examples, the sequence read data 114 can be analyzed (e.g., by the preprocessor 116) to determine endpoint counts of nucleic acid molecule fragments in the sample at multiple genomic positions. In some cases, the sequence read data 114 represents genomic positions in multiple genomic loci. The sequence read data 114 may be limited to genomic positions in one or more genes-of-interest that are relevant for classifying the condition of the subject.

Unprocessed endpoint data 202 indicates the endpoint counts of the nucleic acid molecule fragments at multiple genomic positions. In some examples, the unprocessed endpoint data 202 is indicative of left endpoint positions and/or right endpoint positions of fragments.

Normalized endpoint data 204 is generated, in some examples, by normalizing the unprocessed endpoint data 202. Various sequencing techniques described herein result in different portions of a region being sequenced at different amounts or rates. In particular cases, sequences that correspond to target regions used to generate the endpoint data are sequenced at a higher rate than other sequences. Various bait molecules, for example, are selected within the target region (e.g., a gene or other subgenomic interval-of-interest) in order to enhance the amount of signal obtained in the target region during sequencing. For instance, the sequences that correspond to the bait molecules are tiled (e.g., arranged, with or without interspersed gaps) across the target region. In various cases, the raw endpoint data is normalized based on sequence read data that corresponds to bait molecules used to generate the endpoint data.

Smoothed endpoint data 208 is generated, in various cases, by smoothing the normalized endpoint data 204. In various cases, patterns of endpoint data that are relevant to classification are not necessarily apparent at the single-base level. Therefore, smoothing the endpoint data can enhance the signal-to-noise ratio of the endpoint data without removing potentially relevant endpoint features. In some examples, a smoothing metric may be generated over a window 210 of genomic positions. The window 210, for example, is symmetric at the position. In various cases, the width of the window 210 is in a range of ±3 to ±50 genomic positions around the position. For example, the width of the window 210 is ±5, ±10, ±15, ±30, or ±50 genomic positions around the position. In some cases, the position is assigned as a weighted average of the endpoint counts within the window 210. For example, the smoothed endpoint counts can be generated by convolving, cross-correlating, or multiplying a two-dimensional kernel (e.g., a Gaussian filter) with the endpoint counts in the pre-smoothed fragmentomic data, wherein the two-dimensional kernel itself has the width in the range of ±5 to ±50 genomic positions. Accordingly, in some cases, the smoothed endpoint count at a given position is more dependent on endpoint counts in the center of the window 210 compared to endpoint counts at the edge of the window 210. The value at a given genomic position of the normalized endpoint data 204 is, in various examples, replaced with the smoothing metric of the given genomic position.

Scaled endpoint data 212 is generated, in some examples, based on comparing the smoothed endpoint data 208 to baseline endpoint data 214. In various cases, the scaled endpoint data may be based on the unprocessed endpoint data 202 or the normalized endpoint data 204, rather than the smoothed endpoint data 208. The baseline endpoint data 214 generated based on baseline sequence read data corresponding to baseline subjects. Baseline subjects, in various cases, include individuals who do not have the condition. In some examples, the baseline subjects include individuals with low-shedding tumors (e.g., subjects associated with an absence of ctDNA).

In various cases, a distance metric is calculated for each genomic position based on the baseline endpoint data 214. The distance metric is indicative of the difference between the smoothed endpoint data 208 and the baseline endpoint data 214. For instance, the distance metric may include a z-score that indicates whether the difference between the smoothed endpoint data 208 and the baseline endpoint data 214 at a particular genomic position is statistically significant. The value at each genomic position may be assigned to the distance metric. In some examples, the distance metrics are compared to a threshold, and the scaled endpoint data 212 may indicate the genomic positions associated with a distance metric above the threshold.

In some examples, one or more loci-of-interest are determined by comparing the baseline endpoint data 214 and benchmark endpoint data 216. The benchmark endpoint data 216 is indicative of sequence read data associated with one or more benchmark subjects. The benchmark subject(s), in various cases, include subjects with the condition and/or subjects with particular presentations (e.g., a predetermined subtype) of the condition.

Benchmark metrics 218 are, in some cases, generated based on comparing the baseline endpoint data 214 and the benchmark endpoint data 216. The benchmark metrics 218 may indicate genomic positions having statistical values (e.g., z-scores) that are outside a threshold range (e.g., a confidence interval). These genomic positions, for instance, identify whether the endpoint data of the benchmark samples is abnormal. In various implementations, data derived from genomic positions having statistic values (e.g., z-scores) that are within the threshold range (e.g., the confidence interval) are omitted from further analysis. For instance, the scaled endpoint data 212 may be limited to the genomic positions that are outside the threshold range. Accordingly, the features of the scaled endpoint data 212 that are extracted for further analysis may include, or may be derived from, the portions of the endpoint data that have statistic values outside of the threshold range. The comparison, for instance, can be utilized to reduce the background signal of the endpoint data (e.g., at least one of the unprocessed endpoint data 202, the normalized endpoint data 204, the smoothed endpoint data 208, or the scaled endpoint data 212) of the sample in order to enhance and simplify a subsequent classification process.

According to various implementations, the processed endpoint data 118 described with respect to FIG. 1 may be based on the unprocessed endpoint data 202, the normalized endpoint data 204, the smoothed endpoint data 208, the scaled endpoint data 212, or the benchmark metrics 218. The processed endpoint data 118 may be further analyzed (e.g., by the feature selector 128) in order to determine the condition of the subject.

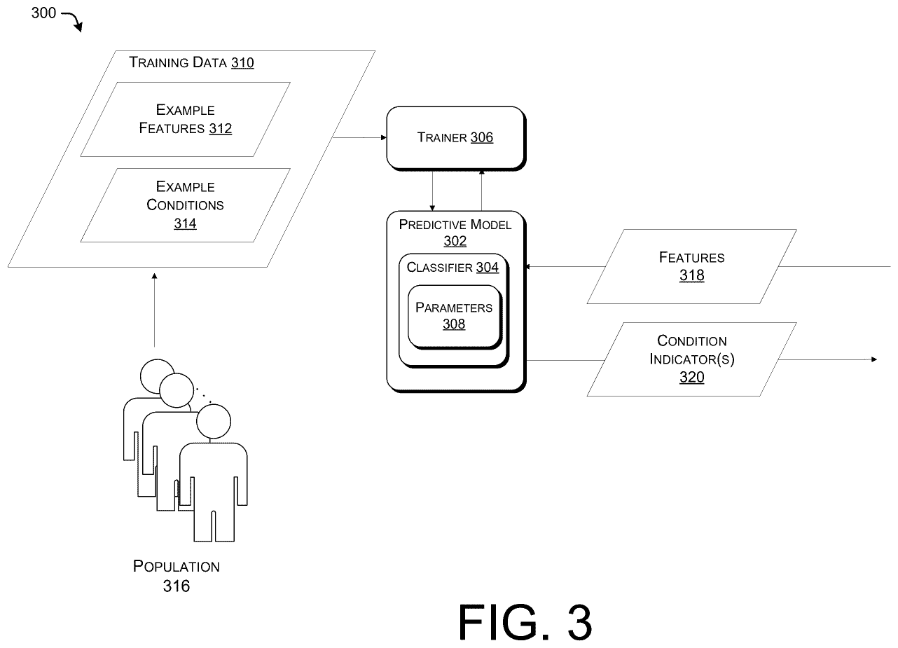

FIG. 3 illustrates an example environment 300 for training and utilizing a predictive model 302 to identify a condition of a subject. The predictive model 302, for instance, is the predictive model 132 described above with reference to FIG. 1. In various implementations, the predictive model 302 includes a classifier 304, which may include one or more ML models. A trainer 306, for instance, is configured to optimize various parameters 308 of the predictive model 302 and/or classifier 304 based on training data 310.

The training data 310 includes example features 312 and example categories 314. The example features 312, in various cases, are obtained based on nucleic acid molecules of individuals within a population 316. In some examples, the example features 312 are obtained based on endpoint data indicated by sequence read data of the nucleic acid molecules. In various cases, the example features 312 are obtained based on preprocessed endpoint data and/or frequency distributions indicative of the endpoint data. The example categories 314 may include categorizations of pathologies (e.g., a cancer type, a cancer subtype, a non-cancer condition, or the like) experienced by the individuals within the population 316. For example, the example categories 314 may be generated based on clinical evaluations of the individuals within the population 316, such as by one or more care providers.

The classifier 304 include one or more model types. For instance, the classifier 304 include an artificial neural network. An artificial neural network includes various layers that respectively process input data. For example, an artificial neural network includes an input layer, one or more hidden layers, and an output layer. The input layer performs a pre-processing operation on the input data. The hidden layer(s) may perform various processing operations on the output from the input layer. The output layer, in various cases, processes the output from the hidden layer(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), convolutional neural networks (CNNs), and backpropagation models. In various implementations, the operations performed by the layers and/or nodes within an artificial neural network included in the classifier 304 is defined according to the parameters 308. For example, the parameters 308 may include weights, thresholds, filters, kernels, or other data objects that are utilized to perform operations of the classifier 304.

In some implementations, the classifier 304 include a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class. For instance, the parameters 308 may include k (e.g., the number of neighbors compared to the new data point), the threshold distance, and so on.

In various cases, the classifier 304 include a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to the parameters 308.

In some cases, the classifier 304 include a clustering model. In various cases, a clustering model maps various data points (e.g., training data) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s) 308, for example, include a threshold proximity within which a new data point is classified within a cluster, a density of points used to define a cluster, and the like.

In various examples, the classifier 304 include a principal component analysis model. In various implementations, a principal component analysis defines a collection of principal components of unit vectors within a coordinate space based on a data set (e.g., training data). The model, for example, is an orthogonal linear transformation of the data set. Various weights of the model, for example, are included in the parameter(s) 308.

The classifier 304, in some implementations, includes a gradient boosting model. For example, the gradient boosting model is defined as a collection of prediction models (e.g., decision trees) that iteratively classify observed data. In various cases, the type of prediction model, weights in the prediction models, and the like, are defined by the parameter(s) 308.

The classifier 304, for example, includes a random forest. The random forest, for instance, includes multiple decision trees that classify data in an ensemble fashion. In various implementations, the decision trees are defined by the parameter(s) 308.

In various implementations of the present disclosure, the trainer 306 is configured to optimize the parameters 308 based on the training data 310. For example, the trainer 306 may input first example features (corresponding to a first individual among the population 316) among the example features 312 into the predictive model 302, and may receive a predicted category. The trainer 306 may compute a loss (e.g., determine a discrepancy) between a first example category (corresponding to the first individual) among the example categories 314 and the predicted category. Further, the trainer 306 may alter the parameters 308 in order to minimize the loss. In various cases, the trainer 306 optimizes the parameters 308 iteratively based on the entire set of the training data 310.

In various implementations, the optimization of the parameters 308 enables the predictive model 302 to identify predictive attributes of the example features 312 that are correlated to or otherwise associated with the example categories 314. For instance, the predictive model 302 may determine that a particular end motif sequence represented in the example features 312 is highly correlated with adenosarcoma. The predictive model 302 may therefore classify cancers based on features outside of the example features 312 by recognizing or otherwise identifying the predictive attributes.

Once the parameters 308 are optimized, the predictive model 302 may be ready to classify a new set of data. For example, the predictive model 302 may receive input data including features 318 (e.g., endpoint data) of a subject. The features 318, for instance, may include one or more of the predictive attributes. The predictive model 302 may perform various operations on the input data based on the trained classifier 304 and the optimized parameters 308. In various cases, the predictive model 302 outputs output data including one or more category indicators 320 based on the features 318. The category indicator(s) 320, for instance, include one or more predicted categories of a cancer experienced by the subject.

Although FIG. 3 is primarily described as referring to supervised learning, implementations are not so limited. In various cases, the training data 310 omits the example categories 314 and the trainer 306 is configured to optimize the parameters 308 using the example features 312 and an unsupervised learning technique.

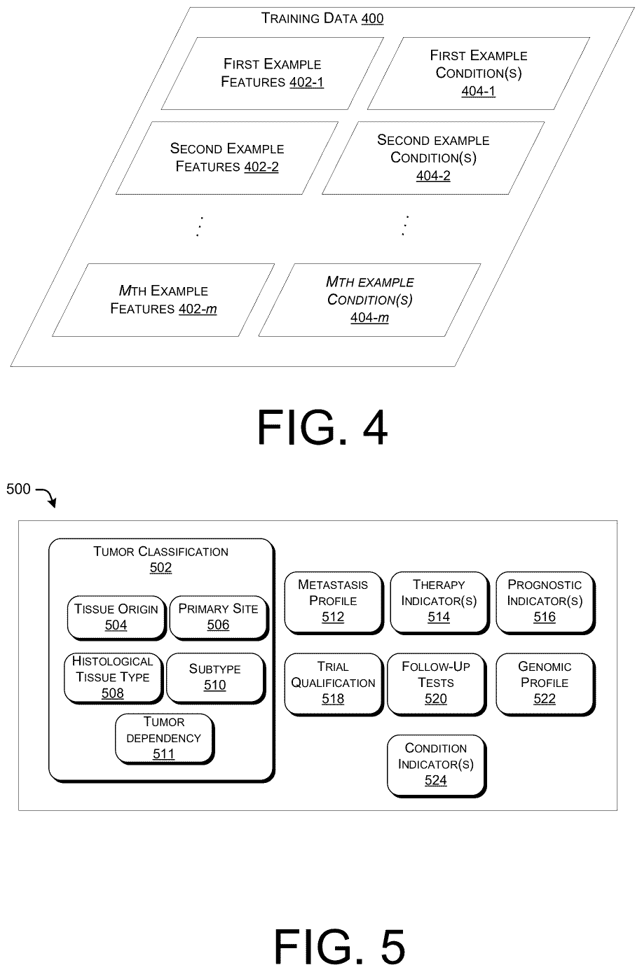

FIG. 4 illustrates an example of training data 400 utilized to train one or more ML models. For example, the training data 400 may be the training data 310 described above with reference to FIG. 3.

The training data 400, in various cases, may represent m samples, wherein m is a positive integer. In some cases, the m samples are respectively obtained from m individuals within a population, although implementations are not so limited. For example, in some cases, multiple samples may be obtained from the same individual at different times.

The training data 400 includes first to mth example features 402-1 to 402-m. For example, the first to mth example features 402-1 to 402-m include features derived from nucleic acid molecules in the respective m samples. In some examples, endpoint data is generated from the nucleic acid molecules detected in the m samples. According to various implementations, the endpoint data is processed by one or more techniques described herein (e.g., normalization, smoothing, scaling) to generate the first to mth example features 402-1 to 402-m. In some cases, spatial domain data is obtained by sequencing the nucleic acid molecules. According to various implementations, the spatial domain data is converted to an alternate domain (e.g., a frequency or wavelet domain) to generate the first to mth example features 402-1 to 402-m. In various cases, the first to mth example features 402-1 to 402-m include fragmentomic features.

The training data 400 may further include first to mth example categories 404-1 to 404-m. The first to mth example categories 404-1 to 404-m, for instance, include categories or classifications of cancers represented by the m samples. In some examples, the first to mth example categories 404-1 to 404-m include tumor classifications of the individuals from which the m samples are obtained. In various cases, the first to mth example categories 404-1 to 404-m include categories or classifications of non-cancer conditions represented by the m samples.

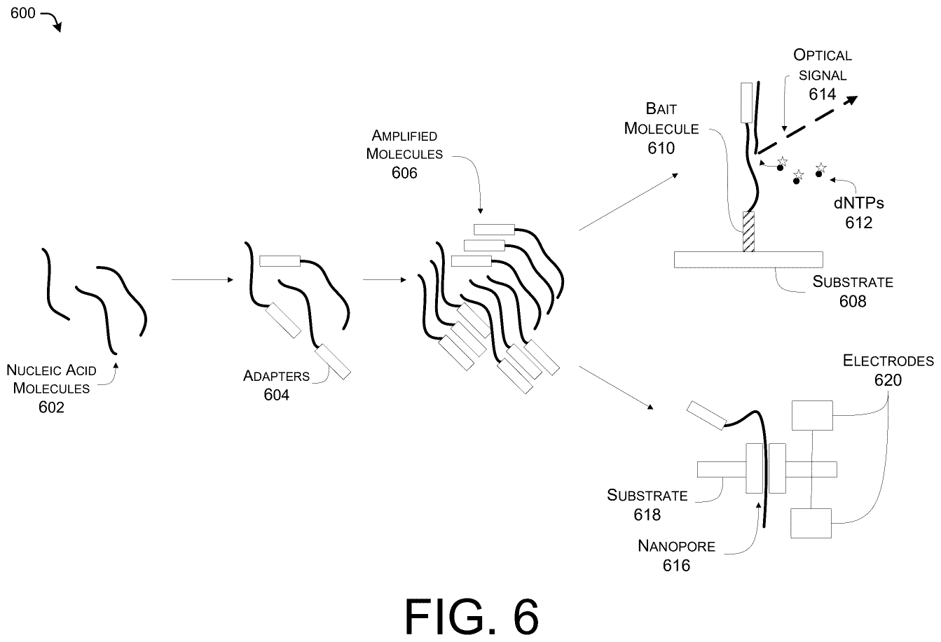

FIG. 5 illustrates an example report 500 summarizing predicted conditions of a subject. In various cases, the report 500 is the report 138 described above with reference to FIG. 1. The report 500, for instance, may be displayed to a patient and/or care provider. In some cases, the report 500 is generated based on features of a sample (e.g., a liquid biopsy sample) obtained from the subject. In various cases, the report 500 is generated based on fragmentomic features of the subject. In various cases, at least some elements of the report 500 are generated based on a predicted classification (e.g., tumor classification, cancer type, etc.) of the subject.

In some cases, the subject is predicted to have a cancer. The report 500 includes a tumor classification 502 of the cancer. The tumor classification 502, in for instance, indicates a tissue origin 504, a primary site 506, a histological tissue type 508, a subtype 510 (e.g., a genomic subtype), a tumor dependency 511, or any combination thereof, of the cancer. The report 500 may include a metastasis profile 512 of the subject. The metastasis profile 512, for instance, indicates a likelihood that the cancer will metastasize (e.g., at a particular point in time), one or more tissues in which the cancer is predicted to metastasize, or the like.

In various cases, the report 500 includes one or more therapy indicators 514. For instance, the therapy indicator(s) 514 convey whether the condition of the subject is predicted to be resistant to one or more predetermined therapies and/or whether the condition of the subject is predicted to be responsive to one or more predetermined therapies.

In some examples, the report 500 includes one or more prognostic indicators 516. The prognostic indicator(s) 516, for instance, indicate a prognosis of the subject in view of the categorized condition. For example, the prognostic indicator(s) 516 may indicate a survivability, a recoverability, a quality of life indicator, or other information indicative of the prognosis of the subject.

The report 500 may include a trial qualification 518 of the subject. The trial qualification 518, for instance, indicates whether the subject is predicted to qualify for a predetermined clinical trial.

In various cases, the report 500 includes recommended follow-up tests 520. For example, the report 500 may include a recommendation to perform whole genome sequencing on the subject (e.g., to sequence the full genome of the subject), particularly in cases if the condition of the subject cannot be categorized above a threshold certainty.

The report 500 may include a genomic profile 522 of the subject. In various cases, the genomic profile 522 includes or is generated based on the results of non-fragmentomic analyses of the subject.

In various implementations, the report 500 includes at least one condition indicator 524. The condition indicator(s) 524, for instance, indicate one or more predicted conditions of the subject. For instance, if the subject is predicted to have a type of cancer, the condition indicator(s) 524 may indicate a cancer type and/or cancer subtype associated with the tumor. In some cases, the condition indicator(s) 524 indicate whether the cancer is associated with particular biomarkers (e.g., hormone receptors, oncogenes, etc.) associated with prognosis and/or susceptibility of the cancer cells to a therapy. In some cases, the condition indicator(s) 524 indicate a non-cancer condition of the subject. The condition indicator(s) 524 may, in some cases, indicate a change in the condition of the subject over time. For instance, the condition indicator(s) 524 may indicate that the cancer of the subject has converted from HR+ to HR-negative. Other types of conditions may also be noted in the condition indicator(s) 524, such as a predicted survivability of the subject, a general health of the subject, a genomic age of the subject, a risk that the subject will develop a disease, a predicted stage of the predicted pathology of the subject, a predicted grade of the predicted pathology of the subject, an ECOG performance status of the subject.

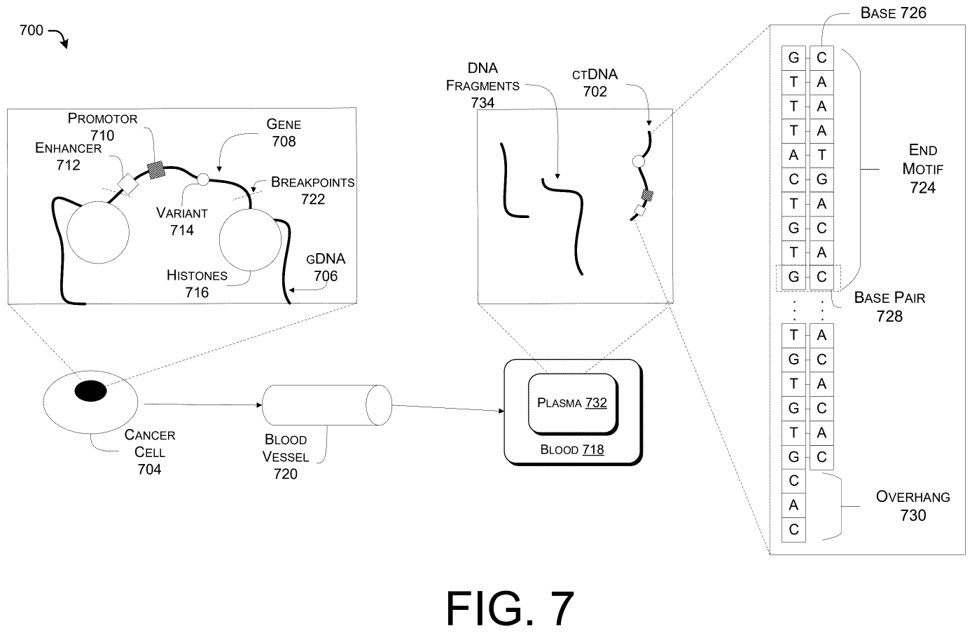

FIG. 6 illustrates an example environment 600 for sequencing various nucleic acid molecules 602. In various implementations, the nucleic acid molecules 602 include cfDNA and/or gDNA. For instance, the nucleic acid molecules 602 may include ctDNA. The nucleic acid molecules 602, in various cases, are extracted from a sample, such as a biological sample obtained from a subject. In some implementations, the nucleic acid molecules 602 include DNA that is complementary to RNA present in the sample.

The nucleic acid molecules 602, in various cases, are ligated with adapters 604. For examples, the adapters 604 are hybridized to the nucleic acid molecules 602. The adapters 604, for example, include additional nucleic acid molecules. In various implementations, the adapters 604 have a shorter length than the nucleic acid molecules 602 being sequenced. For instance, the adapters 604 include amplification primers, flow cell adapter sequences, substrate adapter sequences, or sample index sequences. Although FIG. Y illustrates adapters 604 being ligated to one end of each of the nucleic acid molecules 602, implementations are not so limited. For example, the adapters 604 may be ligated to both ends of each of the nucleic acid molecules 602.

In various examples, the nucleic acid molecules 602 ligated with the adapters 604 are amplified in order to generate amplified molecules 606. Various amplification techniques can be performed. For instance, the amplified molecules 606 are generated using PCR, a non-PCR amplification technique, an isothermal amplification technique, or any combination thereof.

Amplified molecules 606 may be captured by bait molecules 610 and sequenced. In some implementations, the amplified molecules 606 are sequenced via sequencing-by-synthesis. In various cases, fluorescently tagged deoxyribonucleotide triphosphates (dNTP) 612 are utilized to synthesize a strand that is complementary to DNA strands bound to the substrate 608. When a dNTP 612 is added to the strand (e.g., by an enzyme), the dNTP 612 emits an optical signal 614. In various implementations, the frequency of the optical signal 614 is dependent on the type of dNTP 612 from which the optical signal 614 is emitted. By detecting the optical signals 614 as the strand is being synthesized, the sequence of the original nucleic acid molecules 602 can be derived.

In some implementations, the amplified molecules 606 are sequenced via nanopore sequencing. For instance, the amplified molecules 606 are directed through a nanopore 616 extending through a substrate 618. In various cases, the amplified molecules 606 are negatively charged, such that they can be directed through the nanopore 616 by imposing an electrical field across the substrate 618. In various cases, the amplified molecules 606 and the nanopore 616 are in the presence of a charged solution. Thus, charged solutes traveling through the nanopore 616 can be monitored by reviewing an electrical signal (e.g., a current) sensed between electrodes 620 on either side of the substrate 618. As an amplified molecule 606 is directed through the nanopore 616, the individual bases within the amplified molecule 606 will block the nanopore 616, which may decrease the amount of charged solutes traveling through the nanopore 616 and consequently, the magnitude of the electrical signal detected by the electrodes 620. Each of the four types of bases within the amplified molecules 606, may block the nanopore 616 to a different extent. Therefore, the sequence of the nucleic acid molecules 602 can be derived by analyzing the measured electrical signal with respect to time as the amplified molecules 606 are directed through the nanopore 616.

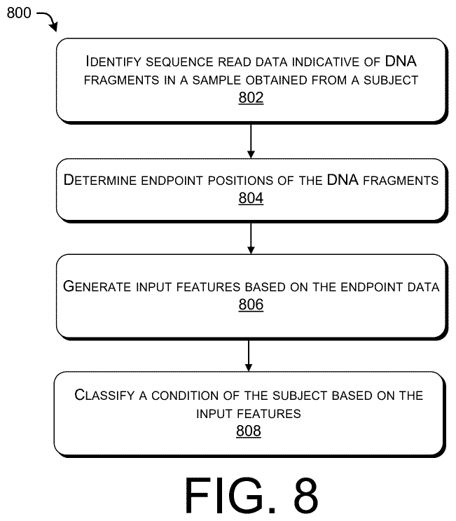

FIG. 7 illustrates an example environment 700 illustrating ctDNA 702, which can be utilized to a condition of a subject. For instance, the ctDNA 702 may be included in the nucleic acid molecules 110 described above with reference to FIG. 1.

In various implementations, a cancer cell 704 within the subject includes genomic DNA (gDNA) that is expressed by the cancer cell 704. For example, the gDNA 706 may include various sequences, such as a gene 708, a promoter 710, an enhancer 712, and a variant 714. For example, the variant 714 is part of the gene 708. In addition, various epigenetic factors impact expression of the gene 708 as well as other genes within the gDNA 706. For example, the gDNA 706 may be packaged within the nucleus of the cancer cell 704 with various histones 716. When the gene 708 is expressed, a portion of the gDNA 706 including the gene 708, the promotor 710, the enhancer 712, and the variant 714 may be exposed to proteins within the nucleus, such as RNA transcriptase. In various cases, the portion of the gDNA 706 is unwrapped or otherwise unpackaged from the histones 716. Thus, the expression of the gene 708 (e.g., the amount of mRNA generated by RNA transcriptase based on the gene 708 within the cancer cell 704) is linked to the frequency or time at which the portion of the gDNA 706 is exposed.

The cancer cell 704, for example, may die. The contents of the cancer cell 704, including the gDNA 706, may be released. In various cases, the gDNA 706 is released into blood 718 that flows through a blood vessel 720 of the subject. When the gDNA 706 is released from the nucleus of the cancer cell 704, the gDNA 706 is degraded due to various biophysical and/or biochemical factors. For example, the blood 718 may include various enzymes that cut the gDNA 706 into the ctDNA 702. In various cases, other mechanical, chemical, or thermal conditions in the blood 718 divide the gDNA 706 into the ctDNA 702. For example, these conditions divide the gDNA 706 into fragments at various breakpoints 722.

Notably, the presence and location of the histones 716 may impact the sequences of the ctDNA 702 that are observed in the blood 718. The breakpoints 722, for example, are more likely to occur at edges of a sequence of the gDNA 706 that is exposed by the histones 716. Therefore, the sequence of the ctDNA 702 is indicative of the expression of mRNA and other functional RNA in the cancer cell 704. By reviewing the ctDNA 702, the expression of the cancer cell 704 can be determined without performing RNA sequencing, in some cases. In various examples, the expression of the cancer cell 704 is relevant to the condition of the subject.

In addition, the sequences at or near the breakpoints 722 are indicative of expression of the cancer cell 704. For example, the ctDNA 702 may include an end motif 724. The end motif 724 may be defined as a sequence of bases 726 and/or base pairs 728 that extend from an end of the ctDNA 702. The end motif 724, for example, has a predetermined length that is in a range of 1 to 30 bases and/or base pairs. In various implementations, the ctDNA 702 is a double-stranded DNA molecule with an overhang 730. The overhang 730, for instance, includes one or more bases 726 of one ssDNA molecule that extends beyond the corresponding end of the other ssDNA molecule. In some cases, the end motif 724 is defined as the sequence of bases in a single ssDNA within the ctDNA 702 or a sequence of complementary base pairs in both ssDNA within the ctDNA 702.

In various implementations, the ctDNA 702 is obtained from a sample of plasma 732 in the blood 718 of the subject. The plasma 732, for example, includes various DNA fragments 734 including the ctDNA 702. In some cases, the DNA fragments 734 include various cfDNA, such as cfDNA released from non-cancerous cells.

By sequencing the ctDNA 702, various fragmentomic features may be obtained. These fragmentomic features can be utilized to categorize the cancer cell 704, thereby identifying a condition of the subject from which the cancer cell 704 was present. In various cases, the fragmentomic features include the presence of at least a portion of the gene 708 in the ctDNA 702. In some cases, the fragmentomic features include the presence of at least a portion of the promotor 710, the enhancer 712, or the variant 714 in the ctDNA 702. In some cases, the fragmentomic features include the presence or sequence of the end motif 724. Other fragmentomic features are described elsewhere herein.

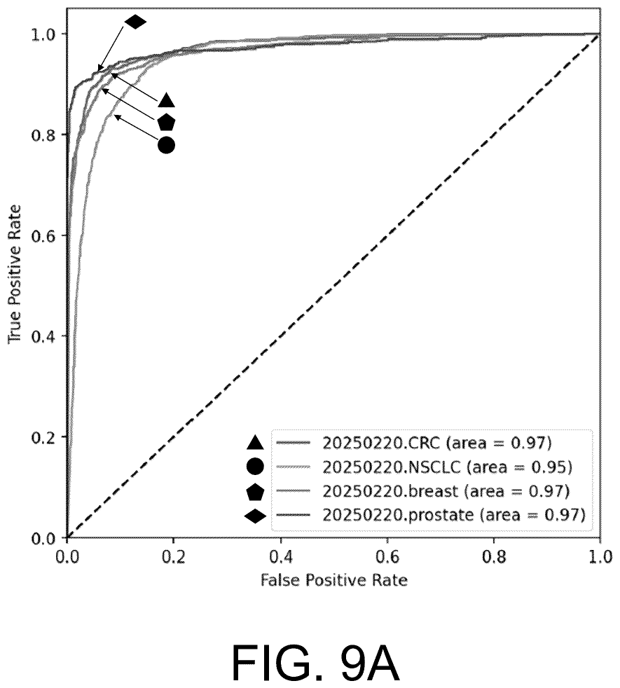

FIG. 8 illustrates an example process 800 for identifying a condition of a subject using fragmentomic data. In various implementations, the process 800 is performed by an entity including at least one processor, at least one computing device, a medical device, the sequencer 112, the preprocessor 116, the feature selector 128, the predictive model 132, the report generator 136, the clinical device 140, the predictive model 302, or any combination thereof.

At 802, the entity identifies sequence read data indicative of DNA fragments of a sample of a subject. The sequence read data, for instance, is indicative of endpoint position. In some cases, the entity generates the sequence read data. For instance, the entity receives a plurality of nucleic acid molecules in a sample from a subject. The sample may include a liquid biopsy sample (e.g., a blood sample, a urine sample, a saliva sample, etc.), a tissue sample, or a combination thereof. The nucleic acid molecules, for instance, include genomic DNA from the sample. One or more adapters are ligated onto at least some of the nucleic acid molecules. The ligated molecules are amplified and captured. In various cases, all or a subset of the captured molecules are sequenced to obtain a plurality of sequence reads that represent the sequenced amplified nucleic acid molecules, thereby generating the sequence read data. In particular examples, the sequence read data includes endpoint counts of DNA fragments at multiple genomic positions within at least one locus of the genome of the sample.

At 804, the entity determines endpoint positions of the DNA fragments with respect to a reference genome. The endpoint positions may include left endpoint positions and/or right endpoint positions of the DNA fragments. In various cases, the entity may determine fragment lengths of the DNA fragments based on, for instance, the left endpoint positions and the right endpoint positions of the DNA fragments. In some examples, the endpoint positions of the DNA fragments are preprocessed. For instance, the endpoint positions of the DNA fragments may be normalized and/or smoothed. In some examples, the endpoint positions of the DNA fragments are scaled by comparing the endpoint positions of the DNA fragments to baseline endpoint positions (e.g., endpoint positions corresponding to samples obtained from individuals who do not have the condition or who have low-shedding tumors associated with the condition). In various instances, the endpoint positions of the DNA fragments are transformed into an alternate domain, before or after preprocessing. In some examples, at least one locus-of-interest is determined by comparing the baseline endpoint positions to benchmark endpoint positions (e.g., endpoint positions corresponding to samples obtained from individuals who have the condition) to identify genomic regions associated with the condition. The endpoint positions of the DNA fragments within the at least one locus-of-interest may be selected for further analysis. In some cases, the entity may generate a frequency distribution indicative of the preprocessed endpoint positions. According to various implementations, the preprocessing may enable identification of features that are indicative of the condition of the subject from the endpoint positions of the DNA fragments.

At 806, the entity determines input features based on the endpoint positions of the DNA fragments. The input features in some examples, are indicative of the condition of the subject. In some implementations, the input features may be based on the sequence read data (e.g., the endpoint positions of the DNA fragments), the preprocessed data (e.g., the preprocessed endpoint positions of the DNA fragments), the transformed data (e.g., the preprocessed endpoint positions of the DNA fragments), or any combination thereof. In some cases, the input features may be based on pre-classified data associated with individuals who do or do not have the condition. In various instances, the input features may be based on an image of the endpoint positions and/or the preprocessed endpoint positions. In various instances, the input features may be based on the left endpoint positions and/or the right endpoint positions of the DNA fragments. In various instances, the input features may be based on the fragment lengths of the DNA fragments. In some examples, the entity may perform a dimensionality reduction technique (e.g., principal component analysis) in order to determine the input features. For instance, the entity may transform training data into a principal component space. The entity may identify principal components that distinguish samples associated with the condition and samples associated with the absence of the condition. These principal components enable extraction of features associated with the condition. The entity may utilize these features to identify input features in the sequence read data and/or the preprocessed data.

At 808, the entity classifies a condition of the subject based on the input features. In some cases, the entity utilized an ML-based classifier to predict whether the subject has the condition. The ML-based classifier, for instance, is pre-trained based on data obtained from a population of individuals that omits the subject. In some cases, the classifier includes at least one of an ANN, a logistic regression model, a decision tree, a KNN model, a support vector machine (SVM), or a naïve Bayes classifier. In some cases, the classifier outputs a likelihood that the subject has a particular condition (or the absence of a particular condition). In some cases, the classifier outputs an indication that the subject has the particular condition (or its absence) when the likelihood exceeds a threshold likelihood.

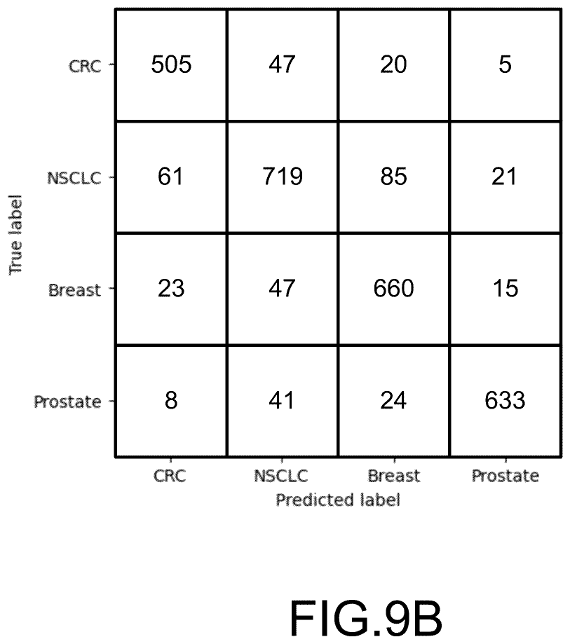

FIGS. 9A and 9B illustrate example classification accuracy using methods described herein. FIG. 9A illustrates the accuracy of an example model configured to determine likelihoods of a sample being associated with colorectal cancer, non-small cell lung cancer (NSCLC), breast cancer, and prostate cancer. FIG. 9B shows the samples stratified by true and predicted labels. As shown in FIGS. 9A and 9B, the disclosed methods can be used to classify samples having tumor fractions equal to or greater than 1% with an area under the curve (AUC) of 0.95 or above.

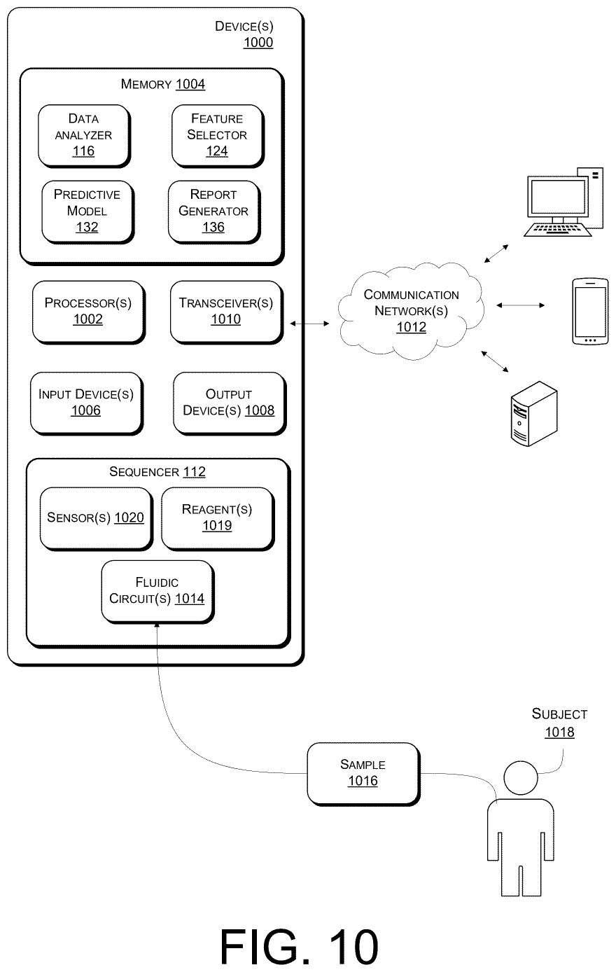

FIG. 10 illustrates one or more devices 1000 configured to perform various operations described herein. The device(s) 1000 include one or more processor(s) 1002. In some implementations, the processor(s) 1002 includes a central processing unit (CPU), a graphics processing unit (GPU), both CPU and GPU, or other processing unit or component known in the art.

The processor(s) 1002 is operably connected to memory 1004. In various implementations, the memory 1004 is volatile (such as random access memory (RAM)), non-volatile (such as read only memory (ROM), flash memory, etc.) or some combination of the two. The memory 1004 stores instructions that, when executed by the processor(s) 1002, causes the processor(s) 1002 to perform various operations. In various examples, the memory 1004 stores methods, threads, processes, applications, objects, modules, any other sort of executable instruction, or a combination thereof. In some cases, the memory 1004 stores files, databases, or a combination thereof. In some examples, the memory 1004 includes, but is not limited to, RAM, ROM, electrically erasable programmable read-only memory (EEPROM), flash memory, or any other memory technology. In some examples, the memory 1004 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 1002. For instance, the memory 1004 stores instructions that, when executed by the processor(s) 1002, causes the processor(s) 1002 to perform operations of the preprocessor 116, the feature selector 128, the predictive model 132, or the report generator 136.

The processor(s) 1002 is operably connected to one or more input devices 1006 and one or more output devices 1008. Collectively, the input device(s) 1006 and the output device(s) 1008 function as an interface between at least one user and the device(s) 1000. The input device(s) 1006 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 1008 includes at least one of a display, a speaker, a haptic output device, a printer, or any combination thereof. In various examples, the processor(s) 1002 causes a display among the input device(s) 1006 to visually output various data described herein. In some implementations, the input device(s) 1006 includes one or more touch sensors, the output device(s) 1008 includes a display screen, and the touch sensor(s) are integrated with the display screen.

In various implementations, the processor(s) 1002 is operably connected to one or more transceivers 1010 that transmit and/or receive data over one or more communication networks 1012. For example, the transceiver(s) 1010 includes a network interface card (NIC), a network adapter, a local area network (LAN) adapter, or a physical, virtual, or logical address to connect to the various external devices and/or systems. In various examples, the transceiver(s) 1010 includes any sort of wireless transceivers capable of engaging in wireless communication (e.g., radio frequency (RF) communication). For example, the communication network(s) 1012 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 1010 includes other wireless modems, such as a modem for engaging in WI-FI®, WIGIG®, WIMAX®, BLUETOOTH®, or infrared communication over the communication network(s) 1012.

The device(s) 1000 may further include the sequencer 112. In various implementations, the sequencer 112 includes one or more fluidic circuits 1014 configured to receive a sample 1016 derived from a subject 1018. The sequencer 112, in various cases, may be configured to generate data indicative of one or more sequences of nucleic acid molecules (e.g., DNA and/or RNA) present in the sample 1016. In various cases, the sequencer 112 introduces one or more reagents 1019 to the fluidic circuit(s) 1014 in order to prepare for and perform sequencing of the nucleic acid molecules. Further, the sequencer 112 may include one or more sensors 1020 disposed on the fluidic circuit(s) 1014 and configured to measure or otherwise detect detection signals from the fluidic circuit(s) 1014, which may be indicative of the sequences of the nucleic acid molecules. According to various implementations, the sensor(s) 1020 may further include one or more ADCs. The sequencer 112, in various cases, outputs sequence read data to the processor(s) 1002 for additional processing.

### Example Clauses

1. A method, including: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, all or a subset of the captured amplified nucleic acid molecules to obtain a plurality of sequence reads that represent the sequenced amplified nucleic acid molecules thereby generating sequence read data; determining, by one or more processors, endpoint counts of fragments indicated by the sequence read data; generating, by the one or more processors, scaled endpoint data representative of the endpoint counts by: normalizing the endpoint counts; smoothing the normalized endpoint counts; and scaling the smoothed endpoint counts based on a plurality of control samples; training, by the one or more processors, a classifier using training data by performing supervised learning, the training data indicating population features of population samples obtained from a population omitting the subject; and determining, using the trained classifier executed by the one or more processors, a tumor classification of the subject based on the scaled endpoint data.
2. The method of clause 1, wherein normalizing the endpoint counts includes:
   normalizing, based on a mean of the endpoint counts, the endpoint counts.
3. The method of clause 1 or 2, wherein smoothing the normalized endpoint counts includes:
   determining a metric over a window of genomic positions centered on an example genomic position of the normalized endpoint counts; and assigning the metric to the example genomic position.
4. The method of clause 3, wherein the metric includes an average endpoint count, a weighted average endpoint count, a median endpoint count, a kernel function, or a filter.
5. The method of any of clauses 1-4, wherein scaling the smoothed endpoint counts based on the plurality of control samples includes: receiving, at the one or more processors, control sequence read data, the control sequence read data being associated with the plurality of control subjects; and determining a distance metric by comparing the smoothed endpoint counts of the fragments to control endpoint counts of the fragments indicated by the control sequence read data.
6. The method of clause 5, wherein the plurality of control subjects are associated with low-shedding tumors.
7. The method of clause 5 or 6, wherein the plurality of control samples have been determined to be free of tumors based on ctDNA tumor fraction estimates of zero.
8. The method of any of clauses 5-7, wherein scaling the smoothed endpoint counts based on the plurality of control samples includes scaling the smoothed endpoint counts into a z-score space based on at least one of the control endpoint counts, a mean of the control endpoint counts, or a standard deviation of the control endpoint counts.
9. The method of any of clauses 5-8, wherein the control sequence read data is first control sequence read data, the control subjects are first control subjects, and the distance metric is a first distance metric, and wherein generating the endpoint data representative of endpoint counts of fragments includes: receiving, at the one or more processors, second control sequence read data, the second control sequence read data being associated with a plurality of second control subjects, the second control subjects having the tumor classification; determining a second distance metric by comparing second control endpoint counts of the fragments indicated by the second control read data to the first control endpoint counts of the fragments indicated by the first control sequence read data; and determining, based on the second distance metric, at least one genomic position associated with the tumor classification of the subject.
10. The method of any of clauses 1-9, wherein generating input features based on the scaled endpoint data includes at least one of: determining, by the one or more processors, principal components indicative of the input features; or inputting, into a machine learning (ML) model configured to detect the input features, the scaled endpoint data.
11. The method of any of clauses 1-10, wherein the tumor classification includes at least one of: a tissue of origin of a cancer of the subject; a histological tissue type of a tumor of the subject; a primary site designation of the tumor of the subject; a tumor dependency of the subject; or a genomic subtype of the cancer of the subject.
12. The method of any of clauses 1-11, wherein the tumor classification includes a likelihood of a cancer subtype of the subject.
13. The method of any of clauses 1-12, wherein the tumor classification includes a first likelihood that the subject has hormone receptor-positive (HR+) breast cancer and a second likelihood that the subject has triple negative (TN) breast cancer.
14. The method of clause 13, wherein the tumor classification further includes a third likelihood that the subject has human epidermal growth factor receptor 2-positive (HER2+) breast cancer.
15. The method of clause 13 or 14, wherein a sum of the first likelihood and the second likelihood is one.
16. A method, including: identifying sequence read data of a sample obtained from a subject; generating endpoint data representative of endpoint counts of DNA fragments indicated by the sequence read data; and classifying, using a classifier, a condition of the subject based on the endpoint data.
17. The method of clause 16, wherein the sequence read data includes endpoint positions of the DNA fragments in the sample at multiple genomic positions.
18. The method of clause 17, wherein the endpoint positions include left endpoint positions and/or right endpoint positions, and wherein the endpoint counts include left endpoint counts and/or right endpoint counts.
19. The method of clause 18, wherein the DNA fragments are located between the left endpoint positions and the right endpoint positions.
20. The method of clause 19, wherein the sequence read data indicates pairs of the left endpoint positions and the right endpoint positions corresponding to each of the DNA fragments.
21. The method of any of clauses 16-20, wherein the sequence read data indicates lengths of the DNA fragments in the sample.
22. The method of any of clauses 16-21, wherein the sequence read data includes read depth of the DNA fragments in the sample at multiple genomic positions.
23. The method of any of clauses 16-22, further including: receiving a plurality of nucleic acid molecules obtained from the sample; ligating one or more adapters onto one or more nucleic acid molecules from the plurality of nucleic acid molecules; amplifying the one or more ligated nucleic acid molecules; capturing all or a subset of the amplified nucleic acid molecules; and sequencing, by a sequencer, the captured nucleic acid molecules to obtain a plurality of sequence reads that represent the captured nucleic acid molecules, thereby generating the sequence read data for a genome of the sample.
24. The method of clause 23, wherein the one or more adapters include amplification primers, flow cell adapter sequences, substrate adapter sequences, or sample index sequences.
25. The method of clause 23 or 24, wherein the captured nucleic acid molecules are captured from the amplified nucleic acid molecules by hybridization to one or more bait molecules.
26. The method of clause 25, wherein the one or more bait molecules include one or more additional nucleic acid molecules, each of the one or more additional nucleic acid molecules including a first region that is complementary to a second region of a captured nucleic acid molecule.
27. The method of any of clauses 23-26, wherein amplifying the one or more ligated nucleic acid molecules includes performing a polymerase chain reaction (PCR) amplification technique, a non-PCR amplification technique, or an isothermal amplification technique.
28. The method of any of clauses 23-27, wherein sequencing the captured nucleic acid molecules includes use of a massively parallel sequencing (MPS) technique, whole genome sequencing (WGS), whole exome sequencing, targeted sequencing, direct sequencing, or Sanger sequencing.
29. The method of any of clauses 23-28, wherein sequencing the captured nucleic acid molecules includes next-generation sequencing (NGS).
30. The method of any of clauses 23-29, wherein the sequencer includes a next-generation sequencer.
31. The method of any of clauses 23-30, wherein sequencing the captured nucleic acid molecules includes sequencing-by-synthesis or nanopore sequencing.
32. The method of any of clauses 16-31, further including: generating ligated molecules by ligating adapters onto nucleic acid molecules of the sample; generating amplified ligated molecules by amplifying the ligated molecules; generating, using the amplified ligated molecules, detection signals; detecting, by at least one sensor, the detection signals; and generating the sequence read data based on the detection signals.
33. The method of clause 32, wherein the detection signals include electrical signals and/or optical signals.
34. The method of clause 32 or 33, wherein generating, using the amplified ligated molecules, the detection signals includes: synthesizing, by a polymerase using fluorescently tagged nucleotide triphosphates (NTPs), a synthesized nucleic acid molecule that is complementary to one of the amplified ligated molecules, and wherein detecting, by the at least one sensor, the detection signals includes: detecting, by at least one optical sensor, optical signals emitted by the fluorescently tagged NTPs upon binding to the synthesized nucleic acid molecule, the optical signals being indicative of at least one sequence of the nucleic acid molecules of the sample.
35. The method of any of clauses 32-34, wherein generating, using the amplified ligated molecules, the detection signals includes: directing the amplified ligated molecules through a nanopore extending from a first space to a second space through a substrate, and wherein detecting, by the at least one sensor, the detection signals includes: detecting, by sensors disposed in the first space and the second space, an electrical signal over time, the electrical signal being indicative of at least one sequence of the nucleic acid molecules of the sample.
36. The method of any of clauses 32-35, wherein the sequence read data indicates a full genome or RNA transcriptome of the sample.
37. The method of any of clauses 32-36, wherein the sequence read data indicates a whole exome of the sample.
38. The method of any of clauses 32-37, wherein the sequence read data indicates a predetermined panel of genes of the sample.
39. The method of clause 38, wherein the predetermined panel includes one or more of A2M, ABCA6, ABCB1, ABCC2, ABCC9, ABI1, ABL1, ABL2, ACACA, ACLY, ACRBP, ACSL3, ACSL6, ACTA2, ACTG1, ACTG2, ACTN1, ACTR3B, ACVR1, ACVR1C, ACVRL1, ADAM12, ADAM19, ADAM2, ADCY7, ADGRB1, ADGRB3, ADGRF5, ADGRL4, ADRB2, AF10, AFF1, AFF3, AFF4, AFP, AGR2, AGR3, AHR, AIFM3, AKT1, AKT2, AKT3, ALDH2, ALK, ALOX12, AMZ1, ANGPT1, ANGPT2, ANLN, ANPEP, ANXA1, ANXA2, APC, APCDD1, APEX2, APH1A, APLN, APOBEC3A, APOBEC3B, APOBR, APOL6, APP, APPBP2, AR, AREG, ARF1, ARG2, ARHGAP15, ARHGDIA, ARID1A, ARID1B, ARID3A, ARNT, ARNT2, ASAP2, ASB13, ASCL2, ASGR2, ASTE1, ASXL1, ATAD2, ATIC, ATM, ATP2C1, ATP8A1, ATP8B2, ATR, AURKA, AURKB, AVPR1A, AXIN2, AXL, B2M, B3GNT5, BAALC, BAG1, BAG2, BAGE4, BAK1, BAMBI, BAP1, BASP1, BATF, BATF3, BAX, BAZ2B, BCAM, BCAR1, BCAR3, BCAS1, BCL10, BCL11A, BCL11B, BCL2, BCL2A1, BCL2L1, BCL2L11, BCL3, BCL6, BCL7A, BCL8, BCL9, BCOR, BCR, BIN2, BIRC3, BIRC5, BLK, BLM, BLNK, BLVRA, BMF, BMP2, BMP4, BMPR1A, BMPR1B, BNC2, BRAF, BRCA1, BRCA2, BRD3, BRD4, BRDT, BRINP3, BRIP1, BRPF1, BTG1, BTG3, BTK, BTLA, BUB1, BUB1B, C10orf35, C11orf30, C15orf48, CIRL, C3, C5, C5AR2, CA4,CAGE1,CALB2, CALML3, CALR, CAMTA1, CANX, CASP1, CASP3, CASP8, CASP9, CBFA2T3, CBFB, CBL, CBLC, CCDC140, CCDC50, CCL11, CCL13, CCL14, CCL17, CCL18,CCL19,CCL2,CCL20,CCL21, CCL3, CCL4, CCL5, CCL8, CCNA2, CCNB1, CCNB2, CCND1,CCND2, CCND3, CCNE1,CCNE2, CCNG2, CCR4, CCR5, CCR7, CCR8, CCRL2, CCSER2, CD14, CD163,CD19, CD1A, CD1B, CD1D, CD1E, CD2,CD209, CD22, CD226, CD244, CD247, CD248, CD27, CD276, CD28, CD33, CD34, CD36, CD38, CD3D, CD3E, CD3G, CD4, CD40, CD40LG, CD44, CD46, CD47,CD5,CD6, CD63, CD68, CD7, CD70, CD74, CD79A, CD79B, CD80, CD81, CD84, CD86,CD8A, CD8B, CD9,CD93, CD96, CDC20, CDC25C, CDC45, CDC6, CDCA3, CDCA5, CDCA7, CDCA7L, CDCA8, CDH1, CDH3,CDH5,CDHR1, CDK2, CDK4, CDK6, CDK8, CDKN1A, CDKN1B, CDKN1C, CDKN2A, CDKN2AIP, CDKN2B, CDKN2B-AS1, CDKN2D, CDKN3, CDT1, CDX2, CEACAM1, CEACAM3, CEACAM5, CEACAM8, CEBPA, CEBPB, CELSR2, CENPA, CENPF, CENPM, CEP110, CEP55, CES1, CES2, CFD, CHAF1B, CHEK1, CHEK2, CHN1, CHUK, CIC, CIITA, CITED4, CLCA2, CLDN18, CLDN3, CLDN4, CLDN5, CLDN6, CLDN7, CLEC10A, CLEC14A, CLEC4C, CLEC5A, CLEC9A, CLIC2, CLIC4, CLTC, CMKLR1, CMPK2, CNN1, CNTNAP2, COL15A1, COL18A1, COL1A1, COL1A2,COL3A1, COL4A1, COL4A2, COL6A3, COL7A1, COPB2, CPA3, CRAT, CREB1, CREB3L1, CREB3L2, CREBBP, CRKL, CRLF2, CRNDE, CRYAB, CSF1, CSF1R, CSF2, CSF3R, CSMD1, CSNK1E, CSNK1G2, CST7,CT45A1, CT45A2, CT45A3, CT62, CTAG1A, CTAG1B, CTAG2, CTAGE1, CTGF, CTLA4, CTNNB1, CTNNBIP1, CTPS1, CTPS2, CTSV, CTSW, CUX1, CX3CL1, CXCL1,CXCL10, CXCL11, CXCL12, CXCL13, CXCL2, CXCL3, CXCL6, CXCL8,CXCL9,CXCR1,CXCR2, CXCR4, CXCR5, CXCR6, CXXC5, CYB5R2, CYBB, CYLD, CYP4F3, DCAF12, DCLK1, DCN, DDB2, DDIT3, DDIT4, DDR1, DDR2, DDX10, DDX21, DDX4, DDX58, DDX6, DEK, DENND3, DEPTOR, DHH, DHX58, DIDO1, DIRC2, DKK1, DKK2, DKK4, DLC1, DLL3, DLL4, DMBT1, DMD, DNMT1, DNMT3A, DOCK5, DOTIL, DRAM1, DSC2, DSCR8, DTL, DTX1, DTX2, DTX3L, DUSP1, DUSP18, DUSP22, DUSP6, DVL1, E2A, E2F1, E2F4, E2F5, EBF1, ECSCR, ECT2, EDNRB, EGF, EGFR, EGLN3, EGR1, EGR2, EIF4A2, ELF4, ELF5, ELK4, ELL, ELN, EMCN, EME1, EML4, EML6, ENL, ENTPD1, EOMES, EP300, EP400, EPCAM, EPHA4, EPHA7, EPOR, EPS15, ERAP1, ERAP2, ERBB2, ERBB3, ERCC1, ERCC2, ERCC3, ERCC4, ERCC5, EREG, ERG, ERN2, ESM1, ESR1, ETO, ETS1, ETV1, ETV4, ETV5, ETV6, EWSR1, EXO1, EZH2, F11R, FAM101B, FAM123B, FAM171B, FAM26F, FAM46A, FAM64A, FANCA, FANCB, FANCC, FANCD2, FAP, FASN, FAT2, FBXW11, FBXW7, FCAR, FCGR2B, FCGR3B, FCRL2, FCRL5, FEV, FGF9, FGFBP2, FGFR1, FGFR10P, FGFR2, FGFR3, FGFR4, FGR, FKBP4, FLI1, FLNA, FLT1, FLT3, FLT3LG, FLT4, FMN1, FMN2, FMOD, FN1, FNBP1, FNIP2, FOLH1, FOLR1, FOS, FOSB, FOXA1, FOXC1, FOXM1, FOX01, FOX03, FOX04, FOX06, FOXP1, FOXP3, FPR1, FPR3, FSTL3, FUCA1, FUS, FUT4, FUT8, FZD1, FZD10, FZD2, FZD5, FZD6, FZD7, GABBR2, GADD45A, GADD45B, GAGE1, GAGE2E, GAGE6, GAGE8, GALNT10, GALNT12, GAS1, GAS7, GBP5, GIMAP5, GIMAP7, GINS2, GJA4, GLI1, GLIS2, GMFG, GMNN, GMPS, GNA12, GNG11, GNLY, GOLM1, GPA33, GPC4, GPC6, GPI, GPR143, GPR146, GPR160, GRAF, GRB7, GREB1, GRM4, GSK3B, GSTA1, GSTM1, GUSB, GZMA, GZMB, GZMH, GZMK, H2AFX, HABP2, HAMP, HAP1, HAVCR2, HBEGF, HCLS1, HCST, HDAC1, HDAC10, HDAC11, HDAC2, HDAC3, HDAC4, HDAC5, HDAC6, HDAC7, HDAC8, HDAC9, HDC, HELZ2, HERPUD1, HES1, HES2, HES4, HES5, HES6, HEY1, HEY2, HEYL, HGF, HHIP, HIF1A, HIP1, HIST1H1A, HIST1H1E, HIST1H2AG, HIST1H2AI, HIST1H2BL, HIST1H3B, HIST2H2BF, HLA-A, HLA-B, HLA-C, HLA-DMA, HLA-DMB, HLA-DOA, HLA-DOB, HLA-DQA1, HLA-DQB1, HLA-DRA, HLA-DRB1, HLA-E, HLF, HMGA1, HMGA2, HMGCS2, HMMR, HOPX, HORMAD1, HOXA11, HOXB2, HPCAL1, HRAS, HRASLS, HSD11B1, HSP90AA1, HSP90AB1, HSPA4L, HSPB1, ICAM1, ICAM2, ICOS, ID1, ID2, IDO1, IFI16, IFI27, IFI35, IFI6, IFIT1, IFIT2, IFIT3, IFITM2, IFITM3, IFNG, IFNL2, IGF1, IGF1R, IGFBP1, IGFBP3, IGFBP4, IGLL5, IHH, IKBKE, IKZF1, IKZF2, IKZF3, IL10, IL11, IL12A, IL13, IL13RA2, IL15, IL16, IL17RA, IL1A, IL1B, IL1R1, IL1RN, IL21R, IL23A, IL2RA, IL3, IL33, IL3RA, ILAR, IL6, IL6R, IL6ST, IL7, IL7R, IMPDH1, INPP1, INSR, INSRR, IPO8, IQGAP3, IRF1, IRF4, IRF7, IRF8, IRGM, IRS2, IRX4, ISG20, ISY1, ITGAM, ITGAV, ITGAX, ITGB1, ITGB2, ITGB4, ITK, ITM2A, ITPKB, JAK1, JAK2, JAK3, JAML, JAZF1, JUN, KCNE3, KCNJ15, KCNK5, KCNMA1, KDM1A, KDM3B, KDM4C, KDM5C, KDM5D, KDR, KDSR, KIAA0040, KIAA0125, KIAA0319L, KIAA1462, KIAA1804, KIF13B, KIF23, KIF2B, KIF2C, KIF5B, KIFC1, KIR2DL1, KIR2DL3, KIR3DL1, KIR3DL2, KIR3DS1, KIT, KLF2, KLF4, KLK3, KLRB1, KLRC3, KLRC4, KLRD1, KLRK1, KMT5A, KRAS, KRT14, KRT17, KRT31, KRT5, KRT6A, KRTCAP3, KYNU, LAG3, LAIR1, LAMB1, LASP1, LATS1, LATS2, LCK, LCN2, LCP1, LDHB, LEF1, LGALS2, LGALS3, LILRB5, LIMD1, LIMK2, LINC-ROR, LINC00598, LIPH, LIPI, LMNA, LMO1, LMO2, LMO3, LMO4, LOC100506207, LOC100507346, LOC100507424, LPP, LRMP, LRP1, LRP8, LRRC15, LTF, LTK, LUZP4, LY6E, LY6G6D, LYL1, LZTR1, MACC1, MAF, MAFB, MAGEA1, MAGEA10, MAGEA11, MAGEA12, MAGEA2B, MAGEA3, MAGEA4, MAGEA5, MAGEA6, MAGEA8, MAGEA9B, MAGEB1, MAGEB10, MAGEB16, MAGEB17, MAGEB18, MAGEB2, MAGEB3, MAGEB4, MAGEB5, MAGEB6, MAGEC1, MAGEC2, MAGEC3, MALAT1, MALT1, MAML2, MAML3, MAP2, MAP2K1, MAP2K3, MAP3K7, MAP3K8, MAP4K4, MAPK1, MAPK3, MAPKAPK2, MAPT, MARK1, MASP2, MAST1, MAST2, MASTL, MB21D1, MBTD1, MCAM, MCL1, MCM10, MCM2, MCM4, MCM6, MDC1, MDM2, MDS2, MECOM, MEF2C, MEF2D, MEG3, MEGF9, MELK, MEN1, MEST, MET, METRNL, MFAP4, MFAP5, MGA, MGMT, MGST2, MIA, MIAT, MICB, MIR100, MITF, MKI67, MKL1, MKL2, MLF1, MLH1, MLL, MLL2, MLL3, MLPH, MME, MMP11, MN1, MNX1, MOCOS, MPZL3, MRAS, MRE11A, MRVI1, MS4A1, MS4A2, MS4A4A, MSH2, MSH6, MSI2, MSMB, MSN, MST1R, MTAP, MTCP1, MTHFD1L, MTOR, MUC1, MUC16, MUTYH, MVP, MX1, MX2, MYB, MYBL2, MYC, MYCL1, MYCN, MYCT1, MYD88, MYH11, MYH9, MYST3, NAB2, NAT1, NAV3, NBEA, NBN, NCAM1, NCOA2, NCOR1, NCR1, NDC80, NDE1, NDRG1, NEAT1, NECTIN1, NECTIN2, NECTIN3, NEK1, NEK2, NEK6, NELL2, NF1, NF2, NFATC2, NFE2L2, NFIC, NFKB2, NID2, NIN, NKD1, NKG7, NKX3-1, NLK, NONO, NOS1, NOS1AP, NOTCH1, NOTCH2, NOTCH3, NOTCH4, NPAS2, NPM1, NR4A3, NRAP, NRARP, NRAS, NRG1, NRG2, NRP1, NRP2, NRTN, NSD1, NT5C3A, NT5E, NTRK1, NTRK2, NTRK3, NUF2, NUMA1, NUMBL, NUP214, NUP98, NUTM1, NUTM2A, NXF2B, NXPH3, OAS3, OASL, ODC1, OGN, OLFM1, OLFM4, OLIG2, ORAI2, ORC6, P2RY8, PADI2, PAFAH1B2, PAGE5, PAK2, PAK4, PALB2, PAMR1, PARP1, PARP12, PARP14, PAX3, PAX5, PAX7, PAX8, PBK, PBX1, PBX3, PCDH17, PCSK1, PDCD1, PDGFA, PDGFB, PDGFD, PDGFRA, PDGFRB, PDIA3, PDL1, PDL2, PDZK1IP1, PECAM1, PFN2, PGR, PHF1, PHF11, PHGDH, PHLPP1, PICALM, PIK3CA, PIK3CD, PIK3CG, PIK3R1, PIK3R2, PIM2, PIM3, PKN1, PLA2G7, PLAC8, PLAG1, PLAGL2, PLCB4, PLEK2, PLEKHA4, PLEKHB1, PLK2, PLPP3, PLVAP, PMEPA1, PML, PMS1, PMS2, PNOC, PNPLA7, PODXL, POLD1, POLE, POU2F2, POU5F1, PPARG, PPM1J, PPP1R13L, PRDM15, PRDM16, PRF1, PRKACA, PRKACB, PRKACG, PRKCA, PRKCB, PRMT1, PRMT5, PRND, PROM1, PRPF6, PRPF8, PSAT1, PSCA, PSD3, PSENEN, PSIP1, PSMB10, PSMB8, PSMB9, PSME1, PTCH1, PTCH2, PTCRA, PTEN, PTGDS, PTGER2, PTGER4, PTGS2, PTPN1, PTPN11, PTPN22, PTPRB, PTPRC, PTPRK, PTPRO, PTPRZ1, PTRF, PTTG1, PUM1, PVR, PVRIG, PXDC1, R3HDM1, RAB23, RAB27A, RAB29, RAC1, RAD50, RAD51, RAD51AP1, RAD51C, RAD51L1, RAD51L3, RAD52, RAD54L, RAF1, RAPGEFL1, RARA, RASGRF1, RASIP1, RASSF6, RB1, RBL1, RBM24, RBP7, RBX1, RECQL4, REG4, RELA, RERG, RET, RGCC, RGS10, RGS16, RGS2, RHOA, RHOH, RHOJ, RIT1, RNF13, ROBO4, ROCK2, ROPN1, ROPN1B, ROR1, RORA, RORC, ROS1, RP1, RPL23, RPL39L, RPS26, RPS6KA1, RPS6KB1, RPSAP52, RRAGC, RRAS, RRM2, RSAD2, RSPO2, RSPO3, RUNDC2A, RUNX1, RUNX2, RUNX3, S100A12, S100A8, S1PR2, SAA1, SAGE1, SAMD9L, SAP30, SCD, SCD5, SCML4, SCUBE2, SDC1, SDHA, SDHB, SDHC, SDHD, SEC31A, SELL, SELP, SEMA3E, SEMA4B, SEMA4C, SEMA6D, SEMA7A, SEPT12, SEPT5, SEPT6, SEPT9, SEPW1, SERPINA9, SERPINB13, SERPINB2, SERPINB5, SERPINE1, SERPINF1, SESN1, SESN2, SESN3, SET, SF3B1, SFRP1, SGK3, SH2D1A, SH2D1B, SH2D2A, SH3BP5, SH3GL1, SH3PXD2A, SHCBP1, SHISA5, SHISA8, SHOC2, SIGLEC5, SKP1, SLAMF1, SLC16A3, SLC1A2, SLC22A8, SLC39A6, SLC40A1, SLC45A3, SLC7A8, SLC9A3R1, SLCO2A1, SLFN11, SLIT2, SMAD2, SMAD3, SMAD4, SMAD9, SMARCB1, SMURF2, SNAI1, SNRNP70, SNW1, SOCS1, SOS1, SOS2,SOX11, SOX17, SOX18, SOX9, SP2, SPANXA1, SPANXB1, SPANXC, SPARC, SPARCL1, SPIB, SPINK1, SPN, SPP1, SPRY4, SRC, SRD5A1, SREBF1, SRSF3, SS18, SSPO, SSX1, SSX2, SSX2B, SSX3, SSX4, SSX5, ST3GAL2, STAT1, STAT3, STAT4, STAT6, STAU2, STEAP1, STEAP4, STIL, STK11, STON1, SULF2, SULT1A1, SUV39H2, SYCP1, SYCP3, SYK, TACSTD2, TAF15, TAGAP, TAGLN, TAL1, TAL2, TAP1, TAP2, TAPBP, TBC1D10C, TBC1D4, TBC1D9, TBL1XR1, TBX21, TCF12, TCF4, TCF7L1, TCF7L2, TCL1, TCL6, TDG, TDGF1, TDRD7, TEAD1, TEC, TEK, TENM3, TERC, TERT, TET1, TET2, TET3, TFCP2L1, TFE3, TFEB, TFF1, TFG, TFPT, TFRC, TGFB1, TGFB2, TGFB3, TGFBI, TGFBR1, TGFBR2, THADA, THBD, THBS1, THY1, TIAM1, TIE1, TIGIT, TIMP3, TLL1, TLR2, TLR3, TLX1, TLX3, TMEM173, TMEM38A, TMEM45B, TMEM55B, TMPRSS2, TNF, TNFRSF10C, TNFRSF11A, TNFRSF14, TNFRSF17, TNFRSF1A, TNFRSF1B, TNFRSF25, TNFRSF6, TNFRSF8, TNFRSF9, TNFSF10, TNFSF11, TNFSF12,TNFSF13B, TNFSF4, TNFSF9, TNKS, TNKS2, TNS1, TOP1, TOP2A, TP53, TP53BP1, TP53INP1, TP53INP2, TP63, TP73, TPM1, TPM2, TPM3, TPM4, TPSAB1, TPSB2, TPST1, TPX2, TRAT1, TREM2, TREX1, TRIM2, TRIM24, TRIM56, TRIP11, TRPS1, TSC1, TSC2, TSHR, TTC39B, TTK, TTL, TTTY14, TTYH1, TWIST1, TYK2, TYMS, UBA7, UBE2C, UBE2T, UBXN4, UGT8, UNC5B, UPK1A, UPP1, USP44, USP6, USP8, VAV3, VCAM1, VCL, VEGFA, VEGFB, VEGFC, VGLL1, VHL, VIM, VNN3, VPREB1, VWF, WASH5P, WBSCR17, WHSC1, WHSC1L1, WIF1, WNT11, WNT16, WNT2, WNT5B, WNT7A, WNT7B, WNT8B, WT1, WWTR1, XCL1, XCL2, XIST, XPA, XPO1, YAP1, YWHAE, YY1, ZAP70, ZBP1, ZBTB16, ZBTB46, ZC3H13, ZC3HAV1, ZEB1, ZEB2, ZIC2, ZMAT3, ZMYM2, ZNF384, ZNF521,ZNF608, ZNF703, ZNF750, or ZNRF3.
40. The method of any of clauses 16-39, wherein the sample includes a tissue biopsy sample, a liquid biopsy sample, or a normal control.
41. The method of clause 40, wherein the sample is the liquid biopsy sample and includes blood, plasma, cerebrospinal fluid, sputum, stool, urine, lymphatic fluid, or saliva.
42. The method of clause 40 or 41, wherein the sample is the liquid biopsy sample and includes circulating tumor cells (CTCs).
43. The method of any of clauses 40-42, wherein the sample is the liquid biopsy sample and includes cfDNA, circulating tumor DNA, or any combination thereof.
44. The method of any of clauses 16-43, wherein the sample is obtained from a tumor of the subject.
45. The method of any of clauses 16-44, further including: receiving the sample.
46. The method of clause 45, further including extracting DNA or RNA from the sample.
47. The method of clause 46, wherein the DNA includes genomic DNA or cDNA.
48. The method of clause 46 or 47, wherein the RNA includes messenger RNA, microRNA, or non-coding RNA.
49. The method of any of clauses 16-48, further including: generating, based on the sequence read data, a frequency distribution of endpoint counts of the DNA fragments indicated by the sequence read data.
50. The method of any of clauses 16-49, wherein generating the endpoint data representative of the endpoint counts includes: normalizing, based on a mean of the endpoint counts within a genomic region, the endpoint counts within the genomic region.
51. The method of clause 50, further including: determining, based on the sequence read data, a ratio of circulating tumor DNA (ctDNA) to cell-free DNA (cfDNA) of the sample; wherein normalizing the endpoint counts includes: normalizing the endpoint counts to the ratio of the ctDNA to the cfDNA of the sample.
52. The method of any of clauses 16-51, wherein generating the endpoint data representative of the endpoint counts includes: smoothing the endpoint counts.
53. The method of clause 52, wherein smoothing the endpoint counts includes: determining a metric over a window of genomic positions centered on an example genomic position of the endpoint counts; and assigning the metric to the example genomic position.
54. The method of clause 53, wherein the metric includes an average endpoint count, a weighted average endpoint count, a median endpoint count, a kernel function, or a filter.
55. The method of clause 53 or 54, wherein the window of genomic positions is in a range of about 2 to about 200 genomic positions.
56. The method of any of clauses 16-55, wherein generating the endpoint data representative the endpoint counts includes: scaling the endpoint counts based on a plurality of control samples.
57. The method of clause 56, wherein scaling the endpoint counts based on a plurality of control samples includes: receiving control sequence read data, the control sequence read data being associated with a plurality of control subjects; and determining a distance metric by comparing the endpoint counts of the DNA fragments to control endpoint counts of DNA fragments indicated by the control sequence read data.
58. The method of clause 57, wherein the plurality of control subjects have a predetermined subtype of the condition and/or have a low-shedding tumor.
59. The method of clause 57 or 58, wherein the plurality of control samples have been determined to be free of tumors based on ctDNA tumor fraction estimates of zero.
60. The method of any of clauses 57-59, wherein the plurality of control subjects lack the condition.
61. The method of any of clauses 57-60, wherein the distance metric is based on: the endpoint counts, and at least one of: the control endpoint counts, a mean of the control endpoint counts, or a standard deviation of the control endpoint counts.
62. The method of clause 61, wherein scaling the endpoint counts based on the plurality of control samples includes: scaling the endpoint counts into a z-score space based on the at least one of the control endpoint counts, the mean of the control endpoint counts, or the standard deviation of the control endpoint counts.
63. The method of any of clauses 57-62, wherein the control sequence read data is first control sequence read data, the control subjects are first control subjects, and the distance metric is a first distance metric, and wherein generating the endpoint data representative of endpoint counts of fragments includes: receiving second control sequence read data, the second control sequence read data being associated with a plurality of second control subjects; determining a second distance metric by comparing second control endpoint counts of DNA fragments indicated by the second control read data to the first control endpoint counts of the DNA fragments indicated by the first control sequence read data; and determining, based on the second distance metric, at least one genomic position associated with the condition of the subject.
64. The method of clause 63, wherein determining the second distance metric includes at least one of: determining a z-score, performing a t-test, or performing a Mann-Whitney U test.
65. The method of clause 63 or 64, wherein the plurality of second control subjects have a predetermined condition.
66. The method of any of clauses 63-65, wherein the second control sequence read data is derived from a plurality of second control samples collected from the plurality of second control subjects, and wherein the plurality of second control samples are associated with non-zero ctDNA tumor fraction estimates.
67. The method of any of clauses 63-66, wherein determining the at least one genomic position associated with the condition includes: comparing the second distance metric to a predetermined threshold.
68. The method of any of clauses 16-67, wherein classifying the condition of the subject includes: generating input features based on the endpoint data; and inputting, to the classifier, the input features.
69. The method of clause 68, wherein generating the input features based on the endpoint data includes at least one of: determining principal components indicative of the input features; or inputting, into a machine learning (ML) model configured to detect the input features, the endpoint data.
70. The method of clause 69, wherein the ML model includes a neural network.
71. The method of clause 70, wherein the neural network includes multiple layers, an individual layer among the multiple layers including a transformation defined by one or more parameters, and wherein extracting the input features from the endpoint data includes generating an output by applying the transformation to the input features, the input features being based on the endpoint data.
72. The method of any of clauses 69-71, wherein the input features are determined based at least in part on pre-classified data, the pre-classified data being generated by: identifying training sequence read data associated with samples corresponding to a plurality of individuals omitting the subject; generating training endpoint data representative of endpoint counts of fragments indicated by the training sequence read data; and generating the pre-classified data by labeling the training endpoint data with labels indicative of conditions of the plurality of individuals.
73. The method of clause 72, further including: training a ML model to identify attributes, indicated by the training endpoint data, that are predictive of the conditions of the plurality of individuals, wherein the input features are instances of the attributes identified via the training of the ML model.
74. The method of any of clauses 68-73, wherein classifying, using the classifier, the condition of the subject based on the input features includes: generating a classification of the condition by inputting, into the classifier, the input features.
75. The method of clause 74, wherein the classifier includes a statistical classifier and/or a machine learning (ML)-based classifier.
76. The method of clause 75, wherein the classifier includes at least one of a: a neural network; a random forest model; or a linear discriminant analysis (LDA) model.
77. The method of clause 75 or 76, wherein the classifier includes at least one of a: a neural network; a logistic regression model; a random forest model; a decision tree; a k-nearest neighbor (KNN) model; a support vector machine (SVM); a naïve Bayes classifier; or a linear discriminant analysis (LDA) model.
78. The method of any of clauses 75-77, wherein the classifier includes a neural network, wherein the neural network includes multiple layers, an individual layer among the multiple layers including a transformation defined by one or more parameters, and wherein the neural network generates an output by applying the transformation to an input, the input being based on the endpoint data and the output being an indication of the condition.
79. The method of any of clauses 75-78, further including training the ML-based classifier based on training data indicative of example DNA fragments identified from example samples of a population.
80. The method of clause 79, wherein the population omits the subject.
81. The method of clause 79 or 80, wherein training the ML-based classifier is based on supervised machine learning, the training data including labels indicating conditions associated with the example samples.
82. The method of clause 81, wherein the ML-based classifier is trained to identify attributes, within the training data, that are predictive of the conditions, and wherein the input features include instances of the attributes identified via the training of the ML-based classifier.
83. The method of any of clauses 79-82, wherein training the ML-based classifier is based on unsupervised machine learning, and wherein training of the ML-based classifier includes identifying a plurality of clusters of the training data.
84. The method of clause 83, further including: identifying at least one cluster, of the plurality of clusters, associated with one or more example samples associated with the condition, wherein the input features are attributes associated with the at least one cluster.
85. The method of any of clauses 16-84, wherein the classifier is configured to provide a binary classification.
86. The method of any of clauses 16-85, wherein the classifier is configured to provide a multi-class classification.
87. The method of any of clauses 16-86, wherein the condition of the subject includes a tumor classification.
88. The method of clause 87, wherein the tumor classification includes at least one of: a tissue of origin of a cancer of the subject; a histological tissue type of a tumor of the subject; a primary site designation of a tumor of the subject; a tumor dependency of the subject; or a genomic subtype of a cancer of the subject.
89. The method of clause 88, wherein the histological tissue type of the tumor includes at least one of: a carcinoma, a sarcoma, a myeloma, a leukemia, or a lymphoma.
90. The method of clause 88 or 89, wherein the histological tissue type of the tumor includes a mixed histological tissue type.
91. The method of clause 90, wherein the mixed histological tissue type includes at least one of: an adenosquamous carcinoma, a carcinosarcoma, a teratocarcinoma, a mixed mesodermal tumor, or a mixed neuroendocrine-non-neuroendocrine neoplasm.
92. The method of any of clauses 88-91, wherein the primary site designation indicates whether the tumor is a primary tumor or a secondary tumor.
93. The method of any of clauses 88-92, wherein the tumor dependency indicates one or more genes and/or proteins associated with survival of a tumor of the subject.
94. The method of any of clauses 87-93, wherein the tumor classification includes a first genomic subtype and a second genomic subtype of a cancer of the subject.
95. The method of any of clauses 87-94, wherein the tumor classification includes a likelihood of a cancer classification of the subject.
96. The method of clause 95, wherein the cancer classification includes a cancer type and/or a cancer subtype.
97. The method of any of clauses 87-96, wherein the tumor classification includes a likelihood that the subject has HR+ breast cancer.
98. The method of any of clauses 87-97, wherein the tumor classification includes a likelihood that the subject has triple negative (TN) breast cancer.
99. The method of any of clauses 87-98, wherein the tumor classification includes a likelihood that the subject has HER2+ breast cancer.
100. The method of any of clauses 87-99, wherein the tumor classification includes a first likelihood that the subject has a first cancer classification and a second likelihood that the subject has a second cancer classification.
101. The method of clause 100, wherein a sum of the first likelihood and the second likelihood is 1.
102. The method of clause 100 or 101, further including: determining, based on the first likelihood and the second likelihood, whether the subject has the first cancer classification, the second cancer classification, or an unknown cancer classification.
103. The method of any of clauses 100-102, wherein the classifier includes a first classifier configured to determine the first likelihood and a second classifier configured to determine the second likelihood.
104. The method of any of clauses 100-103, wherein the tumor classification further includes a third likelihood that the subject has a third cancer classification and a fourth likelihood that the subject has a fourth cancer classification.
105. The method of clause 104, further including: determining, based on the first likelihood, the second likelihood, the third likelihood, and the fourth likelihood, whether the subject has the first cancer classification, the second cancer classification, the third cancer classification, the fourth cancer classification, or an unknown cancer classification.
106. The method of clause 104 or 105, wherein the first cancer classification is non-small cell lung carcinoma, the second cancer classification is breast cancer, the third cancer classification is colorectal cancer, and the fourth cancer classification is prostate cancer.
107. The method of any of clauses 104-106, wherein a sum of the first likelihood, the second likelihood, the third likelihood, and the fourth likelihood is 1.
108. The method of any of clauses 16-107, wherein the condition includes at least one of: a predicted pathologic condition of the subject; a predicted pathologic condition subtype of the subject; a predicted metastasis profile of the subject; a predicted survivability of the subject; a predicted symptom of the subject; a predicted effective therapy to treat the predicted pathologic condition of the subject; a predicted resistance of the subject to a treatment of the predicted pathologic condition; a general health of the subject; a genomic age of the subject; a risk of the subject developing the predicted pathologic condition; a predicted stage of the predicted pathologic condition of the subject; a predicted grade of the predicted pathologic condition of the subject; or a predicted Eastern Cooperative Oncology Group (ECOG) performance status of the subject.
109. The method of any of clauses 16-108, wherein the condition includes a health metric and/or a disease metric of the subject.
110. The method of any of clauses 16-109, wherein the condition includes a likelihood that the subject will develop a disease.
111. The method of any of clauses 16-110, further including: generating, based on the condition, a genomic profile of the subject.
112. The method of clause 111, wherein the genomic profile includes results from at least one of: a comprehensive genomic profiling test; a whole genome sequencing (WGS) test; a whole exome sequencing (WES) test; a gene expression profiling test; a cancer hotspot panel test; a DNA methylation test; a DNA fragmentation test; or an RNA fragmentation test.
113. The method of clause 111 or 112, wherein the genomic profile of the subject includes: results from a nucleic acid sequencing-based test.
114. The method of any of clauses 111-113, further including: selecting, based on the genomic profile and/or the condition, an anticancer agent for administration to the subject.
115. The method of clause 114, further including: administering the anticancer agent to the subject.
116. The method of any of clauses 111-115, further including: applying, based on the genomic profile, an anticancer therapy to the subject.
117. The method of clause 116, wherein the anticancer therapy includes at least one of chemotherapy, radiation therapy, immunotherapy, a targeted therapy, or surgery.
118. The method of any of clauses 111-117, further including: identifying, based on the genomic profile and/or the condition, a suggested treatment decision for the subject.
119. The method of clause 118, wherein the suggested treatment decision includes radiotherapy and/or chemotherapy.
120. The method of any of clauses 111-119, further including: generating a report indicating the genomic profile and/or the condition; and outputting the report.
121. The method of clause 120, wherein outputting the report includes: transmitting data indicating the report to an external device.
122. The method of clause 121, wherein the external device is associated with the subject and/or a healthcare provider.
123. The method of clause 121 or 122, wherein the data is transmitted over one or more communication networks.
124. The method of any of clauses 121-123, wherein the data is transmitted over a peer-to-peer connection.
125. The method of any of clauses 120-124, wherein outputting the report includes: visually presenting, by a display, the report.
126. The method of any of clauses 120-125, further including: determining, based on the genomic profile and/or the condition, one or more therapies to treat the condition of the subject, wherein the report further indicates the one or more therapies.
127. The method of clause 126, wherein the condition includes at least one type or subtype of cancer.
128. The method of any of clauses 111-127, further including: generating, based on the genomic profile and/or the condition, a therapy for the subject.
129. The method of clause 128, wherein the therapy includes a dosage of one or more therapeutic agents predicted to treat the condition of the subject.
130. The method of any of clauses 111-129, further including: determining, based on the genomic profile and/or the condition, whether the subject is eligible for a clinical trial.
131. A system, including: at least one processor; and memory storing instructions that, when executed by the at least one processor, cause the at least one processor to perform operations including: identifying sequence read data of a sample obtained from a subject; generating endpoint data representative of endpoint counts of DNA fragments indicated by the sequence read data; and classifying, using a classifier, a condition of the subject based on the endpoint data.
132. The system of clause 131, further including: a sequencer configured to generate the sequence read data by sequencing a plurality of nucleic acid molecules in the sample.
133. The system of clause 131 or 132, further including: a transceiver configured to transmit data indicating the condition of the subject.
134. The system of any of clauses 131-133, further including: an output device configured to output an indication of the condition of the subject.
135. A non-transitory computer readable medium storing instructions for performing operations including: identifying sequence read data of a sample obtained from a subject; generating endpoint data representative of endpoint counts of DNA fragments indicated by the sequence read data; and classifying, using a classifier, a condition of the subject based on the endpoint data.

### Conclusion

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Tumor mutational burden (TMB) is a measure of the number of mutations carried by tumor cells. By comparing DNA sequences from a patient's healthy tissues and tumor cells, the number of acquired somatic mutations present in tumors, but not in normal tissues, may be determined. In some instances, driver mutations may be excluded from a TMB calculation.

In certain examples, “tumor mutational burden” or “TMB” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In various cases, driver mutations are excluded from a TMB calculation.

Microsatellites are highly polymorphic DNA-repeat regions. In certain examples, “microsatellite” refers to a repetitive nucleic acid having repeat units of less than about 10 base pairs or nucleotides in length. In certain examples, a microsatellite refers to a tract of tandemly repeated (i.e. adjacent) DNA motifs ranging from one to six or up to ten nucleotides, with each motif repeated 5 to 50 repeated times. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy,” Appl Immunohistochem Mol Morphol, 26(2): e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma.

A viral status test refers to a test that identifies the presence of viral RNA or DNA in a subject. The test can identify viral load and/or viral identity. For example, the viral status test can identify the presence of viral RNA or DNA associated with the occurrence of certain cancers. Examples of such viruses include Hepatitis B Virus (HBV) and Hepatitis C Virus (HCV), Kaposi Sarcoma-Associated Herpesvirus (KSHV), Merkel Cell Polyomavirus (MCV), Human Papillomavirus (HPV), Human Immunodeficiency Virus Type 1 (HIV-1, or HIV), Human T-Cell Lymphotropic Virus Type 1 (HTLV-1), and Epstein-Barr Virus (EBV).

Cancer “hotspot” mutations give rise to oncological outcomes. PhyloP, SIFT, Grantham, COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation, EGFR exon 20 activation mutation, EGFR exon 20 insertion, EGFR G719 mutation, EGFR L858R mutation, EGFR L861 mutation, EGFR S768 mutation, EGFR T790M mutation, C797 mutation, KIT activating mutation, KRAS activating mutation, MET activating mutation, NRAS activating mutation, PMS2 promoter mutations, among many others. Hotspot mutations also occur in the following genes: AKT2, BRCA1, BRCA2, ERC1, NSD1, POLH, PPM1G, PTEN, RAD18, RAD51, RAD51B, RB1, TERT, TP53, TP53Bp1, ALK, ARMT1, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1,TACC3, TERF2, TMEM106B, UBE2L3, USP10, WRDR48, YAP1, ZEB2, and ZMYND8.

A “DNA methylation test” refers to an assay, which can be commercially available, for distinguishing methylated versus unmethylated cytosine loci in DNA. Techniques for measuring cytosine methylation include bisulfite-based methylation assays. The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, CA) a quantitative methylation detection assay that uses fluorescence-based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify the target sequence of interest. The amplified PCR product is isolated and used to quantitate the methylation status of the CpG site of interest. (Gonzalgo and Jones Nuclei Acids Res1997; 25:252-31).

In particular embodiments, pyrosequencing can be used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343:249-264. Pyrosequencing is especially useful for detecting methylation in the CpG sites within genes.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Read depth refers to the number of times that a specific genomic site is sequenced during a sequencing run.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A method, comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, all or a subset of the captured amplified nucleic acid molecules to obtain a plurality of sequence reads that represent the sequenced amplified nucleic acid molecules thereby generating sequence read data;
determining, by one or more processors, endpoint counts of fragments indicated by the sequence read data;
generating, by the one or more processors, scaled endpoint data representative of the endpoint counts by:
normalizing the endpoint counts;
smoothing the normalized endpoint counts; and
scaling the smoothed endpoint counts based on a plurality of control samples;
training, by the one or more processors, a classifier using training data by performing supervised learning, the training data indicating population features of population samples obtained from a population omitting the subject; and
determining, using the trained classifier executed by the one or more processors, a tumor classification of the subject based on the scaled endpoint data.

2. (canceled)

3. The method of claim 1, wherein smoothing the normalized endpoint counts comprises:
determining a metric over a window of genomic positions centered on an example genomic position of the normalized endpoint counts; and
assigning the metric to the example genomic position.

4. The method of claim 3, wherein the metric comprises an average endpoint count, a weighted average endpoint count, a median endpoint count, a kernel function, or a filter.

5. The method of claim 1, wherein scaling the smoothed endpoint counts based on the plurality of control samples comprises:
receiving, at the one or more processors, control sequence read data, the control sequence read data being associated with a plurality of control subjects; and
determining a distance metric by comparing the smoothed endpoint counts of the fragments to control endpoint counts of the fragments indicated by the control sequence read data.

6. The method of claim 5, wherein the plurality of control subjects are associated with low-shedding tumors, or
wherein the plurality of control samples have been determined to be free of tumors based on ctDNA tumor fraction estimates of zero.

7. (canceled)

8. The method of claim 5, wherein scaling the smoothed endpoint counts based on the plurality of control samples comprises scaling the smoothed endpoint counts into a z-score space based on at least one of the control endpoint counts, a mean of the control endpoint counts, or a standard deviation of the control endpoint counts.

9-15. (canceled)

16. A method, comprising:
identifying sequence read data of a sample obtained from a subject;
generating endpoint data representative of endpoint counts of DNA fragments indicated by the sequence read data; and
classifying, using a classifier, a condition of the subject based on the endpoint data.

17. The method of claim 16, wherein the sequence read data comprises left endpoint positions and/or right endpoint positions of the DNA fragments in the sample at multiple genomic positions, and wherein the endpoint counts comprise left endpoint counts and/or right endpoint counts.

18. (canceled)

19. (canceled)

20. The method of claim 17, wherein the sequence read data indicates pairs of the left endpoint positions and the right endpoint positions corresponding to each of the DNA fragments and/or lengths of the DNA fragments in the sample.

21-35. (canceled)

36. The method of claim 16, wherein the sequence read data indicates a full genome or RNA transcriptome of the sample,
wherein the sequence read data indicates a whole exome of the sample, or
wherein the sequence read data indicates a predetermined panel of genes of the sample.

37-48. (canceled)

49. The method of claim 16, further comprising:
generating, based on the sequence read data, a frequency distribution of the endpoint counts of the DNA fragments indicated by the sequence read data.

50. The method of claim 16, wherein generating the endpoint data representative of the endpoint counts comprises one or more of:
normalizing, based on a mean of the endpoint counts within a genomic region, the endpoint counts within the genomic region;
smoothing the endpoint counts; or
scaling the endpoint counts based on a plurality of control samples.

51-67. (canceled)

68. The method of claim 16, wherein classifying the condition of the subject comprises:
generating input features based on the endpoint data; and
inputting, to the classifier, the input features.

69. The method of claim 68, wherein generating the input features based on the endpoint data comprises at least one of:
determining principal components indicative of the input features; or
inputting, into a machine learning (ML) model configured to detect the input features, the endpoint data.

70-84. (canceled)

85. The method of claim 16, wherein the classifier is configured to provide a binary classification, or
wherein the classifier is configured to provide a multi-class classification.

86. (canceled)

87. (canceled)

88. The method of claim 16, wherein the condition of the subject comprises a tumor classification, the tumor classification comprising at least one of:
a tissue of origin of a cancer of the subject;
a histological tissue type of a tumor of the subject;
a primary site designation of the tumor of the subject;
a tumor dependency of the subject;
a genomic subtype of the cancer of the subject;
a first likelihood of a cancer classification of the subject; or
a second likelihood that the subject has a first cancer classification and a third likelihood that the subject has a second cancer classification.

89-119. (canceled)

120. The method of claim 16, further comprising:
generating a report indicating the condition; and
outputting the report.

121-127. (canceled)

128. The method of claim 16, further comprising:
generating, based on the condition, a therapy for the subject; and/or
determining, based on the condition, whether the subject is eligible for a clinical trial.

129. The method of claim 128, wherein the therapy comprises a dosage of one or more therapeutic agents predicted to treat the condition of the subject.

130. (canceled)

131. A system, comprising:
at least one processor; and
memory storing instructions that, when executed by the at least one processor, cause the at least one processor to perform operations comprising:
identifying sequence read data of a sample obtained from a subject;
generating endpoint data representative of endpoint counts of DNA fragments indicated by the sequence read data; and
classifying, using a classifier, a condition of the subject based on the endpoint data.

132-135. (canceled)
